SEQUENCE LISTING (25 sequences)

SEQ ID NO: 1 - DNA, 1251 bp, Homo sapiens
ggatggggagataagagaagaccaggagggagttaaatagggaatgggttgggggcggcttggtaaatgtgctgggattaggctgttgcagataatgcaacaaggcttggaaggctaacctgggactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatcaaaaaa

SEQ ID NO: 2 - DNA, 1220 bp, Homo sapiens
gggacagggggagccctataattggacaagtctgggatccttgagtcctactcagccccagcggaggtgaaggacgtccttccccaggagccgactggccaatcacaggcaggaagatgaaggttctgtgggctgcgttgctggtcacattcctggcaggatgccaggccaaggtggagcaagcggtggagacagagccggagcccgagctgcgccagcagaccgagtggcagagcggccagcgctgggaactggcactgggtcgcttttgggattacctgcgctgggtgcagacactgtctgagcaggtgcaggaggagctgctcagctcccaggtcacccaggaactgagggcgctgatggacgagaccatgaaggagttgaaggcctacaaatcggaactggaggaacaactgaccccggtggcggaggagacgcgggcacggctgtccaaggagctgcaggcggcgcaggcccggctgggcgcggacatggaggacgtgtgcggccgcctggtgcagtaccgcggcgaggtgcaggccatgctcggccagagcaccgaggagctgcgggtgcgcctcgcctcccacctgcgcaagctgcgtaagcggctcctccgcgatgccgatgacctgcagaagcgcctggcagtgtaccaggccggggcccgcgagggcgccgagcgcggcctcagcgccatccgcgagcgcctggggcccctggtggaacagggccgcgtgcgggccgccactgtgggctccctggccggccagccgctacaggagcgggcccaggcctggggcgagcggctgcgcgcgcggatggaggagatgggcagccggacccgcgaccgcctggacgaggtgaaggagcaggtggcggaggtgcgcgccaagctggaggagcaggcccagcagatacgcctgcaggccgaggccttccaggcccgcctcaagagctggttcgagcccctggtggaagacatgcagcgccagtgggccgggctggtggagaaggtgcaggctgccgtgggcaccagcgccgcccctgtgcccagcgacaatcactgaacgccgaagcctgcagccatgcgaccccacgccaccccgtgcctcctgcctccgcgcagcctgcagcgggagaccctgtccccgccccagccgtcctcctggggtggaccctagtttaataaagattcaccaagtttcacgcatcaaaaa

SEQ ID NO: 3 - protein, 317 aa, Homo sapiens [sequence omitted]

SEQ ID NO: 4 - DNA, 954 bp, Artificial Sequence
  Synthetic polynucleotide
atgaaggtgctgtgggccgccctgctggtgaccttcctggccggctgccaggccaaagtcgaacaggccgtcgagaccgagcccgagcccgagctgcgccagcagaccgagtggcagagcggccagcgctgggagctggccctgggccgcttctgggactacctgcgctgggtgcagaccctgagcgagcaggtgcaggaggagctgctgagcagccaggtgacccaggagctgcgcgccctgatggacgagaccatgaaagaactcaaagcttataagagcgagctggaggagcagctgacccccgtggccgaggagacccgcgcccgcctgagcaaggagctgcaggccgcccaggcccgcctgggcgccgacatggaggacgtgtgcggccgcctggtgcagtaccgcggcgaggtgcaggccatgctgggccagagcaccgaggagctgcgcgtgcgcctggccagccacctgcgcaagctgcgcaagcgcctgctgcgcgacgccgacgacctgcagaagtgcctggccgtgtaccaggccggcgcccgcgagggcgccgagcgcggcctgagcgccatccgcgagcgcctgggccccctggtggagcagggccgcgtgcgcgccgccaccgtgggcagcctggccggccagcccctgcaggagcgcgcccaggcctggggcgagcgcctgcgcgcccgcatggaggagatgggcagccgcacccgcgaccgcctggacgaggtgaaggagcaggtggccgaggtgcgcgccaagctggaggagcaggcccagcagatccgcctgcaggccgaggccttccaggcccgcctgaagagctggttcgagcccctggtggaggacatgcagcgccagtgggccggcctggtggagaaggtgcaggccgccgtgggcaccagcgccgcccccgtgcccagcgacaaccactaa

SEQ ID NO: 5 - DNA, 21 bp, Artificial Sequence
  Synthetic polynucleotide
ttgtaggccttcaactccttc

SEQ ID NO: 6 - DNA, 21 bp, Artificial Sequence
  Synthetic polynucleotide
gaaggagttgaaggcctacaa

SEQ ID NO: 7 - DNA, 58 bp, Artificial Sequence
  Synthetic polynucleotide
ttgtaggccttcaactccttccatctgtggcttcactgaaggagttgaaggcctacaa

SEQ ID NO: 8 - DNA, 152 bp, Artificial Sequence
  Synthetic polynucleotide
ttgtcatcctcccacggtggccatttgttccatgtgagtgctagtaacaggccttgtgtcctttgtaggccttcaactccttccatctgtggcttcactgaaggagttgaaggcctacaagacaacagcatacagccttcagcaagcctcca

SEQ ID NO: 9 - DNA, 903 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagcaaaaaaattgtcatcctcccacggtggccatttgttccatgtgagtgctagtaacaggccttgtgtcctttgtaggccttcaactccttccatctgtggcttcactgaaggagttgaaggcctacaagacaacagcatacagccttcagcaagcctccagtggtctcatacagaacttataagattcccaaatccaaagacatttcacgtttatggtgatttcccagaacacatagcgacatgcaaatattgcagggcgccactcccctgtccctcacagccatcttcctgccagggcgcacgcgcgctgggtgttcccgcctagtgacactgggcccgcgattccttggagcgggttgatgacgtcagcgtttcccatggtgaagcttggatctgatccctaggttctagaaccggtgaccaattgttaattaagtttaaaccctcgaggccgcaagcagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 10 - DNA, 2551 bp, Artificial Sequence
  Synthetic polynucleotide
ttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtacctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtcttgtaggccttcaactccttcagtgaagccacagatggaaggagttgaaggcctacaaaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccacccaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 11 - DNA, 3506 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtcttgtaggccttcaactccttcagtgaagccacagatggaaggagttgaaggcctacaaaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgaaggtgctgtgggccgccctgctggtgaccttcctggccggctgccaggccaaagtcgaacaggccgtcgagaccgagcccgagcccgagctgcgccagcagaccgagtggcagagcggccagcgctgggagctggccctgggccgcttctgggactacctgcgctgggtgcagaccctgagcgagcaggtgcaggaggagctgctgagcagccaggtgacccaggagctgcgcgccctgatggacgagaccatgaaagaactcaaagcttataagagcgagctggaggagcagctgacccccgtggccgaggagacccgcgcccgcctgagcaaggagctgcaggccgcccaggcccgcctgggcgccgacatggaggacgtgtgcggccgcctggtgcagtaccgcggcgaggtgcaggccatgctgggccagagcaccgaggagctgcgcgtgcgcctggccagccacctgcgcaagctgcgcaagcgcctgctgcgcgacgccgacgacctgcagaagtgcctggccgtgtaccaggccggcgcccgcgagggcgccgagcgcggcctgagcgccatccgcgagcgcctgggccccctggtggagcagggccgcgtgcgcgccgccaccgtgggcagcctggccggccagcccctgcaggagcgcgcccaggcctggggcgagcgcctgcgcgcccgcatggaggagatgggcagccgcacccgcgaccgcctggacgaggtgaaggagcaggtggccgaggtgcgcgccaagctggaggagcaggcccagcagatccgcctgcaggccgaggccttccaggcccgcctgaagagctggttcgagcccctggtggaggacatgcagcgccagtgggccggcctggtggagaaggtgcaggccgccgtgggcaccagcgccgcccccgtgcccagcgacaaccactaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 12 - DNA, 21 bp, Artificial Sequence
  Synthetic polynucleotide
ctccaccgcttgctccacctt

SEQ ID NO: 13 - DNA, 21 bp, Artificial Sequence
  Synthetic polynucleotide
aaggtggagcaagcggtggag

SEQ ID NO: 14 - DNA, 58 bp, Artificial Sequence
  Synthetic polynucleotide
ctccaccgcttgctccaccttagtgaagccacagatgaaggtggagcaagcggtggag

SEQ ID NO: 15 - DNA, 152 bp, Artificial Sequence
  Synthetic polynucleotide
tggaggcttgctgaaggctgtatgctgttgtcctccaccgcttgctccaccttagtgaagccacagatgaaggtggagcaagcggtggagaggacacaaggcctgttactagcactcacatggaacaaatggccaccgtgggaggatgacaa

SEQ ID NO: 16 - DNA, 3506 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtcctccaccgcttgctccaccttagtgaagccacagatgaaggtggagcaagcggtggagaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgaaggtgctgtgggccgccctgctggtgaccttcctggccggctgccaggccaaagtcgaacaggccgtcgagaccgagcccgagcccgagctgcgccagcagaccgagtggcagagcggccagcgctgggagctggccctgggccgcttctgggactacctgcgctgggtgcagaccctgagcgagcaggtgcaggaggagctgctgagcagccaggtgacccaggagctgcgcgccctgatggacgagaccatgaaagaactcaaagcttataagagcgagctggaggagcagctgacccccgtggccgaggagacccgcgcccgcctgagcaaggagctgcaggccgcccaggcccgcctgggcgccgacatggaggacgtgtgcggccgcctggtgcagtaccgcggcgaggtgcaggccatgctgggccagagcaccgaggagctgcgcgtgcgcctggccagccacctgcgcaagctgcgcaagcgcctgctgcgcgacgccgacgacctgcagaagtgcctggccgtgtaccaggccggcgcccgcgagggcgccgagcgcggcctgagcgccatccgcgagcgcctgggccccctggtggagcagggccgcgtgcgcgccgccaccgtgggcagcctggccggccagcccctgcaggagcgcgcccaggcctggggcgagcgcctgcgcgcccgcatggaggagatgggcagccgcacccgcgaccgcctggacgaggtgaaggagcaggtggccgaggtgcgcgccaagctggaggagcaggcccagcagatccgcctgcaggccgaggccttccaggcccgcctgaagagctggttcgagcccctggtggaggacatgcagcgccagtgggccggcctggtggagaaggtgcaggccgccgtgggcaccagcgccgcccccgtgcccagcgacaaccactaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 17 - DNA, 19 bp, Artificial Sequence
  Synthetic polynucleotide
tttgtaggccttcaactcc

SEQ ID NO: 18 - DNA, 19 bp, Artificial Sequence
  Synthetic polynucleotide
ggagttgaaggcctacaaa

SEQ ID NO: 19 - DNA, 54 bp, Artificial Sequence
  Synthetic polynucleotide
tttgtaggccttcaactccagtgaagccacagatgggagttgaaggcctacaaa

SEQ ID NO: 20 - DNA, 148 bp, Artificial Sequence
  Synthetic polynucleotide
tggaggcttgctgaaggctgtatgctgttgtctttgtaggccttcaactccagtgaagccacagatgggagttgaaggcctacaaaaggacacaaggcctgttactagcactcacatggaacaaatggccaccgtgggaggatgacaa

SEQ ID NO: 21 - DNA, 3502 bp, Artificial Sequence
  Synthetic polynucleotide
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgctagctctgggtatttaagcccgagtgagcacgcagggtctccattttgaagcgggaggttacgcgttcgtcgactactagtgggtaccagagctccctaggttctagaaccggtgacgtctcccatggtgaagcttggatctgaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctcagcgctgtaattagcgcttggtttaatgacggcttgttggaggcttgctgaaggctgtatgctgttgtctttgtaggccttcaactccagtgaagccacagatgggagttgaaggcctacaaaaggacacaaggcctgttactagcactcacatggaacaaatggccaccgtgggaggatgacaatttctgtggctgcgtgaaagccttgaggggctccgggagctagagcctctgctaaccatgttcatgccttcttctttttcctacagctcctgggcaacgtgctggttattgtgctgtctcatcattttggcaaagaattcctcgaagatccgaagggaaagtcttccacgactgtgggatccgttcgaagatatcaccggttgagccaccatgaaggtgctgtgggccgccctgctggtgaccttcctggccggctgccaggccaaagtcgaacaggccgtcgagaccgagcccgagcccgagctgcgccagcagaccgagtggcagagcggccagcgctgggagctggccctgggccgcttctgggactacctgcgctgggtgcagaccctgagcgagcaggtgcaggaggagctgctgagcagccaggtgacccaggagctgcgcgccctgatggacgagaccatgaaagaactcaaagcttataagagcgagctggaggagcagctgacccccgtggccgaggagacccgcgcccgcctgagcaaggagctgcaggccgcccaggcccgcctgggcgccgacatggaggacgtgtgcggccgcctggtgcagtaccgcggcgaggtgcaggccatgctgggccagagcaccgaggagctgcgcgtgcgcctggccagccacctgcgcaagctgcgcaagcgcctgctgcgcgacgccgacgacctgcagaagtgcctggccgtgtaccaggccggcgcccgcgagggcgccgagcgcggcctgagcgccatccgcgagcgcctgggccccctggtggagcagggccgcgtgcgcgccgccaccgtgggcagcctggccggccagcccctgcaggagcgcgcccaggcctggggcgagcgcctgcgcgcccgcatggaggagatgggcagccgcacccgcgaccgcctggacgaggtgaaggagcaggtggccgaggtgcgcgccaagctggaggagcaggcccagcagatccgcctgcaggccgaggccttccaggcccgcctgaagagctggttcgagcccctggtggaggacatgcagcgccagtgggccggcctggtggagaaggtgcaggccgccgtgggcaccagcgccgcccccgtgcccagcgacaaccactaacaattgttaattaagtttaaaccctcgaggccgcaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatcgataccgtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatccacgataacaaacagcttttttggggtgaacatattgactgaattccctgcaggttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 22 - DNA, 19 bp, Artificial Sequence
  Synthetic polynucleotide
tattagatctgatggccgc

SEQ ID NO: 23 - DNA, 20 bp, Artificial Sequence
  Synthetic polynucleotide
ctccatcactaggggttcct

SEQ ID NO: 24 - DNA, 60 bp, Artificial Sequence
  Synthetic polynucleotide
agctctgggtatttaagcccgagtgagcacgcagggtctccattttgaagcgggaggtta

SEQ ID NO: 25 - DNA, 145 bp, Artificial Sequence
  Synthetic polynucleotide
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

(19) United States
(12) Patent Application Publication
(10) Pub. No.: US 2022/0010001 A1
(43) Pub. Date: Jan. 13, 2022

(54) GENE THERAPIES FOR NEURODEGENERATIVE DISEASE

(71) Applicant: Prevail Therapeutics, Inc., New York, NY (US)

(72) Inventors: Asa Abeliovich, New York, NY (US); Laura Heckman, New York, NY (US); Herve Rhinn, New York, NY (US)

(73) Assignee: Prevail Therapeutics, Inc., New York, NY (US)

(21) Appl. No.: 17/297,492

(22) PCT Filed: Nov. 26, 2019
(86) PCT No.: PCT/US2019/063289

§ 371 Date: May 27, 2021

Related U.S. Application Data
(60) Provisional application No. 62/772,230, filed on Nov. 28, 2018.

(51) Int. Cl. C07K 14/775; C12N 15/113; C12N 15/86; A61P 25/28

(52) U.S. Cl. CPC C07K 14/775; C12N 15/113; A61K 48/00; A61P 25/28; C12N 2750/14143; C12N 15/86

(57) ABSTRACT

The disclosure relates, in some aspects, to compositions and methods for treatment of neurodegenerative disease, for example Alzheimer's disease. In some embodiments, the disclosure provides expression constructs comprising a transgene encoding an APOE protein isoform or a portion thereof, an inhibitory nucleic acid targeting an APOE gene or a portion thereof, or any combination of the foregoing. In some embodiments, the disclosure provides methods of treating Alzheimer's disease by administering an expression construct to a subject in need thereof.

### RELATED APPLICATIONS

This Application is a national stage filing under 35 U.S.C. § 371 of international application PCT/US2019/063289, filed Nov. 26, 2019, which claims priority under 35 U.S.C. § 119(e) to U.S. provisional patent application Ser. No. 62/772,230, filed Nov. 28, 2018, the entire contents of each of which are incorporated herein by reference.

### BACKGROUND

Alzheimer's disease (AD) is the most common form of dementia, affecting more than 5 million people in the United States alone. Alzheimer's disease is an irreversible, progressive brain disorder characterized by the presence of abnormal protein deposits throughout the brain, which inhibit neuronal function, disrupt connections between neurons, and ultimately result in cell death. These deposits comprise plaques of amyloid-β and tangles formed by phosphorylated-tau proteins. Patients with mild AD experience memory loss, leading to wandering, difficulty handling money, repeating questions, and personality and behavior changes. Moderate AD patients exhibit increased memory loss, leading to confusion and difficulty recognizing friends and family, inability to learn new things, hallucinations, delusions, and paranoia. Patients with severe AD cannot communicate and are completely depending on others for their care. Ultimately, protein plaques and tangles spread throughout the brain, leading to significant tissue shrinkage.

### SUMMARY

Most Alzheimer's disease (AD) patients have late-onset AD, in which symptoms being to appear in the subject's mid-60's. The apolipoprotein E (APOE) gene is involved in the development of late-onset AD. APOE has several isoforms, including APOE2, which is protective against AD, and APOE4, which is associated with increased risk for developing late-onset AD. Homozygous patients who carry two copies of APOE4 (e.g., subjects that are APOE4^{+/+}) are at an even greater risk of developing late-onset AD as compared to heterozygous patients who carry one copy of APOE4 and one copy of either APOE2 or APOE3.

Aspects of the disclosure relate to compositions and methods for treating a subject having or suspected of having AD. The disclosure is based, in part, on expression constructs encoding an inhibitory RNA (e.g., shRNA, miRNA, amiRNA, etc.) that targets an AD-associated gene (e.g., APOE, such as APOE4).

In some aspects, the disclosure is based on expression constructs (e.g., vectors) encoding APOE2 (or a portion thereof) and, optionally, one or more additional gene products from AD-associated genes (e.g., an inhibitory nucleic acid that targets APOE4). Without wishing to be bound by any particular theory, combinations of gene products described herein act together (e.g., synergistically) to reduce one or more signs and symptoms of AD when expressed in a subject.

Accordingly, in some aspects, the disclosure provides an isolated nucleic acid comprising an expression construct encoding an APOE2 protein, wherein the isolated nucleic acid comprises the sequence set forth in SEQ ID NO: 4.

In some embodiments, the disclosure provides an isolated nucleic acid comprising an expression construct encoding an inhibitory nucleic acid that inhibits expression or activity of APOE4 and a transgene that expresses APOE2. In some embodiments, the expression construct is flanked by adeno-associated virus (AAV) inverted terminal repeats (ITRs). In some embodiments, the ITRs are AAV2 ITRs.

In some embodiments, an inhibitory nucleic acid is complementary to at least six contiguous nucleotides of the sequence set forth in SEQ ID NO: 1. In some embodiments, an inhibitory nucleic acid is an inhibitory RNA comprising (or encoded by) the nucleic acid sequence set forth in any one of SEQ ID NOs: 5-8, 12-15, and 17-20. In some embodiments, an inhibitory nucleic acid comprises (or is encoded by) the sequence set forth in any one of SEQ ID NOs: 7, 8, 14, 15, 19, and 20.

In some embodiments, a transgene that expresses APOE2 encodes a protein having an amino acid sequence set forth in SEQ ID NO: 3. In some embodiments, a transgene that expresses APOE2 comprises a codon optimized nucleic acid sequence. In some embodiments, a codon-optimized nucleic acid sequence encoding APOE2 is set forth in SEQ ID NO: 4.

In some embodiments, the disclosure provides an isolated nucleic acid comprising the sequence set forth in any one of SEQ ID NOs: 11, 16, and 21.

In some aspects, the disclosure provides an isolated nucleic acid comprising an expression construct encoding an APOE2 protein, wherein the isolated nucleic acid comprises the sequence set forth in SEQ ID NO: 4. In some aspects, the disclosure provides an isolated nucleic acid comprising an expression construct encoding an inhibitory nucleic acid that inhibits expression or activity of APOE4. In some embodiments, the expression construct is flanked by adeno-associated virus (AAV) inverted terminal repeats (ITRs), optionally wherein the ITRs are AAV2 ITRs.

In some embodiments, an isolated nucleic acid further comprises one or more promoters. In some embodiments, a promoter is a chicken-beta actin (CBA) promoter, a CAG promoter, a CD68 promoter, or a JeT promoter.

In some embodiments, the disclosure provides a vector comprising an isolated nucleic acid as described by the disclosure. In some embodiments, a vector is a plasmid. In some embodiments, a vector is a viral vector. In some embodiments, a viral vector is a recombinant AAV (rAAV) vector or a Baculovirus vector.

In some aspects, the disclosure provides a composition comprising an isolated nucleic or a vector as described herein. In some embodiments, the disclosure provides a host cell comprising an isolated nucleic acid or a vector as described herein.

In some aspects, the disclosure provides a recombinant adeno-associated virus (rAAV) comprising: a capsid protein; and an isolated nucleic acid or a vector as described herein.

In some embodiments, a capsid protein is capable of crossing the blood-brain barrier. In some embodiments, a capsid protein is an AAV9 capsid protein or an AAVrh.10 capsid protein. In some embodiments, an rAAV transduces neuronal cells and non-neuronal cells of the central nervous system (CNS).

In some aspects, the disclosure provides a method for treating a subject having or suspected of having Alzheimer's disease, the method comprising administering to the subject an isolated nucleic acid, a vector, a composition, or an rAAV as described by the disclosure.

In some embodiments, administration comprises direct injection to the CNS of the subject. In some embodiments, direct injection is intracerebral injection, intraparenchymal injection, intrathecal injection, or any combination thereof. In some embodiments, direct injection to the CNS of the subject comprises convection enhanced delivery (CED). In some embodiments, administration comprises peripheral injection. In some embodiments, peripheral injection is intravenous injection. In some embodiments, a subject is homozygous for APOE4 alleles (e.g., APOE4+/+).

### BRIEF DESCRIPTION OF THE DRAWINGS

FIG. 1 is a schematic depicting one embodiment of a plasmid comprising an rAAV vector that includes an expression construct encoding an inhibitory RNA targeting APOE (e.g., APOE transcript variant 4 ApoE4). The inhibitory RNA is operably linked to an H1 promoter.

FIG. 2 is a schematic depicting one embodiment of a plasmid comprising an rAAV vector that includes an expression construct encoding an inhibitory RNA targeting APOE (e.g., APOE transcript variant 4 ApoE4). The inhibitory RNA is positioned within an intron and is operably linked to a promoter sequence.

FIG. 3 is a schematic depicting one embodiment of a plasmid comprising an rAAV vector that includes an expression construct encoding an inhibitory RNA targeting APOE (e.g., APOE transcript variant 4 ApoE4). The inhibitory RNA is positioned within an intron between a promoter sequence and an APOE2 protein-encoding sequence.

FIG. 4 is a schematic depicting one embodiment of a plasmid comprising an rAAV vector that includes an expression construct encoding an inhibitory RNA targeting APOE (e.g., APOE transcript variant 4 ApoE4). The inhibitory RNA is positioned within an intron between the promoter sequence and an APOE2 protein-encoding sequence.

FIG. 5 is a schematic depicting one embodiment of a plasmid comprising an rAAV vector that includes an expression construct encoding an inhibitory RNA targeting APOE (e.g., APOE transcript variant 4 ApoE4). The inhibitory RNA is positioned within an intron between the promoter sequence and an APOE2 protein-encoding sequence.

FIG. 6 is a schematic depicting an rAAV vectors comprising a “D” region located on the “outside” of the ITR (e.g., proximal to the terminus of the ITR relative to the transgene insert or expression construct) (top) and a wild-type rAAV vectors having ITRs on the “inside” of the vector (e.g., proximal to the transgene insert of the vector).

FIGS. 7A and 7B show in vitro validation of rAAV vectors carrying different shRNAs against APOE4, and a codon optimized APOE2 coding sequence, by qRT-PCR. FIG. 7A shows that several candidate vectors successfully reduced endogenous APOE expression. FIG. 7B shows that the shRNAs expressed by these vectors do not affect the expression of the codon-optimized APOE2.

FIG. 8 is a schematic depicting the experimental design of in vivo selection of rAAV vectors carrying different shRNAs against APOE4 and codon optimized APOE2 coding sequence using APOE4 knock-in mice.

### DETAILED DESCRIPTION

The disclosure is based, in part, on compositions and methods for expression of combinations of AD-associated gene products in a subject. A gene product can be a protein, a fragment (e.g., portion) of a protein, an interfering nucleic acid that inhibits an AD-associated gene, etc. In some embodiments, a gene product is a protein or a protein fragment encoded by an AD-associated gene. In some embodiments, a gene product is an interfering nucleic acid (e.g., shRNA, siRNA, miRNA, amiRNA, etc.) that inhibits an AD-associated gene.

An AD-associated gene refers to a gene encoding a gene product that is genetically, biochemically or functionally associated with Alzheimer's disease (AD). For example, individuals having at least one copy of APOE4 are at an increased risk of developing late-onset AD. In another example, APOE2 exhibits a neuroprotective effect in mouse models of AD. As used herein, the term “neuroprotective” refers to the preservation of neuronal structure and/or function in a cell or subject relative to the preservation of neuronal structure and/or function in a cell or subject in the absence of neuroprotection (e.g., the absence of a neuroprotective agent or protein).

### Isolated Nucleic Acids and Vectors

An isolated nucleic acid may be DNA or RNA. In some aspects, the disclosure provides an isolated nucleic acid comprising an expression construct encoding an inhibitory nucleic acid targeting APOE4 and/or a transgene encoding an APOE2 protein or a portion thereof.

Generally, an isolated nucleic acid as described herein may encode 1, 2, 3, 4, 5, 6, 7, 8, 9, 10, or more inhibitory nucleic acids (e.g., dsRNA, siRNA, shRNA, miRNA, amiRNA, etc.). In some embodiments, an isolated nucleic acid encodes more than 10 inhibitory nucleic acids. In some embodiments, each of the one or more inhibitory nucleic acids targets a different gene or a portion of a gene (e.g., a first miRNA targets a first target sequence of a gene and a second miRNA targets a second target sequence of the gene that is different than the first target sequence). In some embodiments, each of the one or more inhibitory nucleic acids targets the same target sequence of the same gene (e.g., an isolated nucleic acid encodes multiple copies of the same miRNA).

Aspects of the disclosure relate to an isolated nucleic acid comprising an expression construct encoding one or more interfering nucleic acids (e.g., dsRNA, siRNA, miRNA, amiRNA, etc.) that target an APOE4 protein (e.g., isoform E4 of the APOE gene). APOE protein refers to apolipoprotein E, which is a fat binding protein that plays a role in catabolism of triglyceride-rich lipoproteins. There are three major isoforms of APOE, referred to as APOE2, APOE3, and APOE4. Each isoform differs from the others at two positions, amino acid 130 and amino acid 176 (also respectively referred to as positions 112 and 158 when the signal peptide of the protein is excluded). APOE2 contains Cys130/Cys176 and has been observed to be associated with type III hyperlipoproteinemia and other diseases but also plays a neuroprotective role. APOE3 contains Cys130/Arg176 and is the most common APOE allele. APOE4 contains Arg130/Arg176 and has been observed to be associated with late-onset Alzheimer's disease, atherosclerosis, unfavorable outcomes in traumatic brain injury (TBI) and other diseases. In humans, APOE gene is located on chromosome 19. In some embodiments, APOE4 is encoded by a nucleic acid sequence set forth in SEQ ID NO: 1 (e.g., NCBI Reference Sequence Number NM_001302690.1). In some embodiments, the APOE2 is encoded by a nucleic acid sequence set forth in SEQ ID NO: 2 (e.g., NCBI Reference Sequence Number NM_000041.3).

An inhibitory nucleic acid targeting APOE gene (e.g., APOE4) may comprise a region of complementarity (e.g., a region of the inhibitory nucleic acid that hybridizes to the target gene, for example a gene encoding APOE4) that is between 6 and 50 nucleotides in length. In some embodiments, an inhibitory nucleic acid comprises a region of complementarity with APOE that is between about 6 and 30, about 8 and 20, or about 10 and 19 nucleotides in length. In some embodiments, an inhibitory nucleic acid is complementary with at least 3, 4, 5, 6, 7, 8, 9, 10, 11, 12, 13, 14, 15, 16, 17, 18, 19, 20, 21, 22, 23, 24, or 25 contiguous nucleotides of a APOE sequence. In some embodiments, an inhibitory nucleic acid targeting an APOE gene is non-allele-specific (e.g., the inhibitory nucleic acid silences all isoforms of APOE gene). In some embodiments, an inhibitory nucleic acid targets one or more specific alleles of APOE, for example one or more of APOE2, APOE3, and/or APOE4.

In some embodiments, a gene product (e.g., a transgene encoding APOE2) is encoded by a coding portion (e.g., a cDNA) of a naturally occurring gene. In some embodiments, a gene product is a protein (or a fragment thereof) encoded by the APOE2 isoform of the APOE gene. In some embodiments, an APOE2 gene comprises the nucleic acid sequence set forth in SEQ ID NO: 3. In some embodiments, a gene product is an inhibitory nucleic acid that targets (e.g., hybridizes to, or comprises a region of complementarity with) an AD-associated gene (e.g., APOE4 isoform of the APOE gene). A skilled artisan recognizes that the order of expression of a first gene product (e.g., APOE2) and a second gene product (e.g., inhibitory RNA targeting APOE4 isoform of the APOE gene) can generally be reversed (e.g., the inhibitory RNA is the first gene product and APOE2 is the second gene product). In some embodiments, a gene product is a fragment (e.g., portion) of an APOE gene. A protein fragment may comprise about 50%, about 60%, about 70%, about 80% about 90% or about 99% of a protein encoded by an APOE gene. In some embodiments, a protein fragment comprises between 50% and 99.9% (e.g., any value between 50% and 99.9%) of a protein having the amino acid sequence set forth in SEQ ID NO: 3. In some embodiments, a gene product (e.g., an inhibitory RNA) hybridizes to portion of a target gene (e.g., is complementary to 5, 6, 7, 8, 9, 10, 11, 12, 13, 14, 15, 16, 17, 18, 19, 20, 21, or more contiguous nucleotides of a target gene, for example APOE4 isoform of APOE, such as the sequence set forth in SEQ ID NO: 1).

In some embodiments, an expression construct is monocistronic (e.g., the expression construct encodes a single fusion protein comprising a first gene product and a second gene product). In some embodiments, an expression construct is polycistronic (e.g., the expression construct encodes two distinct gene products, for example two different proteins or protein fragments).

A polycistronic expression vector may comprise a one or more (e.g., 1, 2, 3, 4, 5, or more) promoters. Any suitable promoter can be used, for example, a constitutive promoter, an inducible promoter, an endogenous promoter, a tissue-specific promoter (e.g., a CNS-specific promoter), etc. In some embodiments, a promoter is a chicken beta-actin promoter (CBA promoter), a CAG promoter (for example as described by Alexopoulou et al. (2008) BMC Cell Biol. 9:2; doi: 10.1186/1471-2121-9-2), a CD68 promoter, or a JeT promoter (for example as described by Tornøe et al. (2002) Gene 297(1-2):21-32). In some embodiments, a promoter is operably-linked to a nucleic acid sequence encoding a first gene product, a second gene product, or a first gene product and a second gene product. In some embodiments, an expression cassette comprises one or more additional regulatory sequences, including but not limited to transcription factor binding sequences, intron splice sites, poly(A) addition sites, enhancer sequences, repressor binding sites, or any combination of the foregoing.

In some embodiments, a nucleic acid sequence encoding a first gene product and a nucleic acid sequence encoding a second gene product are separated by a nucleic acid sequence encoding an internal ribosomal entry site (IRES). Examples of IRES sites are described, for example, by Mokrejs et al. (2006) Nucleic Acids Res. 34(Database issue):D125-30. In some embodiments, a nucleic acid sequence encoding a first gene product and a nucleic acid sequence encoding a second gene product are separated by a nucleic acid sequence encoding a self-cleaving peptide. Examples of self-cleaving peptides include but are not limited to T2A, P2A, E2A, F2A, BmCPV 2A, and BmIFV 2A, and those described by Liu et al. (2017) Sci Rep. 7: 2193. In some embodiments, the self-cleaving peptide is a T2A peptide.

In some embodiments, disorders such as AD are associated with the expression of at least one copy of APOE4. Accordingly, in some embodiments, isolated nucleic acids described herein comprise an inhibitory nucleic acid that reduces or prevents the expression of APOE4 (e.g., APOE). A sequence encoding an inhibitory nucleic acid may be placed in an untranslated region (e.g., intron, 5′UTR, 3′UTR, etc.) of an expression vector.

In some embodiments, an inhibitory nucleic acid is positioned in an intron of an expression construct, for example in an intron upstream of the sequence encoding a first gene product. An inhibitory nucleic acid can be a double stranded RNA (dsRNA), shRNA, siRNA, micro RNA (miRNA), artificial miRNA (amiRNA), or an RNA aptamer. Generally, an inhibitory nucleic acid binds to (e.g., hybridizes with) between about 6 and about 30 (e.g., any integer between 6 and 30, inclusive) contiguous nucleotides of a target RNA (e.g., mRNA). In some embodiments, the inhibitory nucleic acid molecule is an miRNA or an amiRNA, for example an miRNA that targets the APOE4 isoform of APOE (the gene encoding APOE4 protein). In some embodiments, the miRNA does not comprise any mismatches with the region of APOE mRNA to which it hybridizes (e.g., the miRNA is “perfected”). In some embodiments, the inhibitory nucleic acid is an shRNA (e.g., an shRNA targeting APOE), for example as set forth in any one of SEQ ID NOs: 7, 14, and 19. In some embodiments, an miRNA comprises at least one (e.g., 1, 2, 3, 4, 5, 6, 7, 8, 9, 10, or more) mismatches with the region of APOE mRNA to which it hybridizes.

In some embodiments, an inhibitory nucleic acid is an artificial microRNA (amiRNA). A microRNA (miRNA) typically refers to a small, non-coding RNA found in plants and animals and functions in transcriptional and post-translational regulation of gene expression. MiRNAs are transcribed by RNA polymerase to form a hairpin-loop structure referred to as a pri-miRNAs which are subsequently processed by enzymes (e.g., Drosha, Pasha, spliceosome, etc.) to for a pre-miRNA hairpin structure which is then processed by Dicer to form a miRNA/miRNA* duplex (where * indicates the passenger strand of the miRNA duplex), one strand of which is then incorporated into an RNA-induced silencing complex (RISC). In some embodiments, an inhibitory RNA as described herein is a miRNA targeting the APOE4 isoform of APOE (the gene encoding APOE4 protein).

In some embodiments, an inhibitory nucleic acid targeting APOE (e.g., the APOE4 isoform of APOE) comprises a miRNA/miRNA* duplex. In some embodiments, the miRNA strand of a miRNA/miRNA* duplex comprises or consists of the sequence set forth in any one of SEQ ID NOs: 5, 6, 12, 13, 17, and 18. In some embodiments, the miRNA* strand of a miRNA/miRNA* duplex comprises or consists of the sequence set forth in any one of SEQ ID NOs: 5, 6, 12, 13, 17, and 18.

An artificial microRNA (amiRNA) is derived by modifying native miRNA to replace natural targeting regions of pre-mRNA with a targeting region of interest. For example, a naturally occurring, expressed miRNA can be used as a scaffold or backbone (e.g., a pri-miRNA scaffold), with the stem sequence replaced by that of an miRNA targeting a gene of interest. An artificial precursor microRNA (pre-amiRNA) is normally processed such that one single stable small RNA is preferentially generated. In some embodiments, rAAV vectors and rAAVs described herein comprise a nucleic acid encoding an amiRNA. In some embodiments, the pri-miRNA scaffold of the amiRNA is derived from a pri-miRNA selected from the group consisting of pri-MIR-21, pri-MIR-22, pri-MIR-26a, pri-MIR-30a, pri-MIR-33, pri-MIR-122, pri-MIR-375, pri-MIR-199, pri-MIR-99, pri-MIR-194, pri-MIR-155, and pri-MIR-451. In some embodiments, an amiRNA comprises a nucleic acid sequence targeting APOE (e.g., APOE4 isoform of APOE) and an eSIBR amiRNA scaffold, for example as described in Fowler et al. Nucleic Acids Res. 2016 March 18; 44(5): e48.

In some embodiments, an amiRNA targeting APOE (e.g., APOE4 isoform of APOE) comprises or consists of the sequence set forth in any one of SEQ ID NOs: 8, 15, and 20.

An isolated nucleic acid as described herein may exist on its own, or as part of a vector. Generally, a vector can be a plasmid, cosmid, phagemid, bacterial artificial chromosome (BAC), or a viral vector (e.g., adenoviral vector, adeno-associated virus (AAV) vector, retroviral vector, baculoviral vector, etc.). In some embodiments, the vector is a plasmid (e.g., a plasmid comprising an isolated nucleic acid as described herein). In some embodiments, the vector is a recombinant AAV (rAAV) vector. An rAAV may comprise either the “plus strand” or the “minus strand” of an rAAV vector. In some embodiments, an rAAV vector is single-stranded (e.g., single-stranded DNA). In some embodiments, a vector is a Baculovirus vector (e.g., an Autographa californica nuclear polyhedrosis (AcNPV) vector).

Typically an rAAV vector comprises a transgene (e.g., an expression construct comprising one or more of each of the following: promoter, intron, enhancer sequence, protein coding sequence, inhibitory RNA coding sequence, polyA tail sequence, etc.) flanked by two AAV inverted terminal repeat (ITR) sequences. In some embodiments the transgene of an rAAV vector comprises an isolated nucleic acid as described by the disclosure. In some embodiments, each of the two ITR sequences of an rAAV vector is a full-length ITR (e.g., approximately 145 bp in length, and containing functional Rep binding site (RBS) and terminal resolution site (trs)). In some embodiments, one of the ITRs of an rAAV vector is truncated (e.g., shortened or not full-length). In some embodiments, a truncated ITR lacks a functional terminal resolution site (trs) and is used for production of self-complementary AAV vectors (scAAV vectors). In some embodiments, a truncated ITR is a AITR, for example as described by McCarty et al. (2003) Gene Ther. 10(26):2112-8.

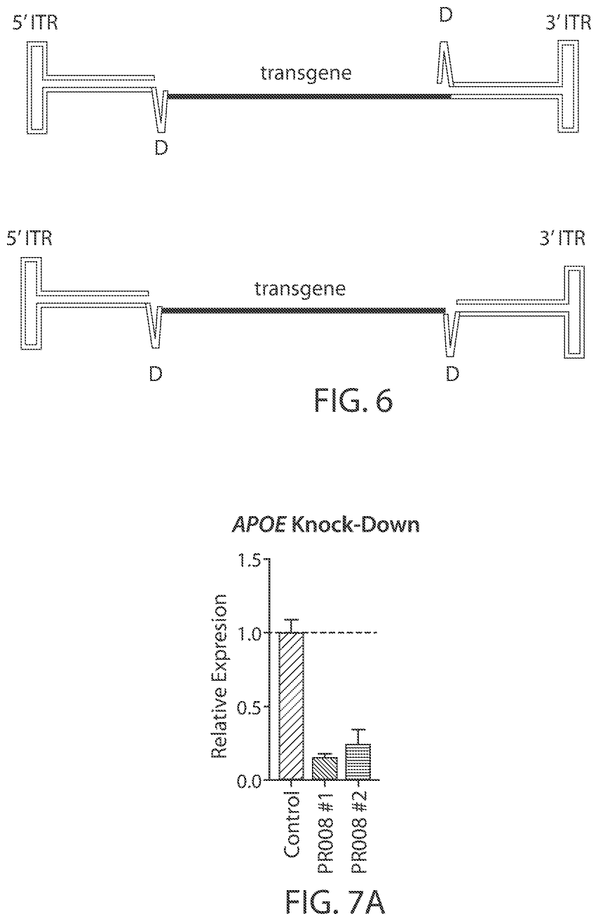

Aspects of the disclosure relate to isolated nucleic acids (e.g., rAAV vectors) comprising an ITR having one or more modifications (e.g., nucleic acid additions, deletions, substitutions, etc.) relative to a wild-type AAV ITR, for example relative to wild-type AAV2 ITR (e.g., SEQ ID NO: 25). The structure of wild-type AAV2 ITR is shown in FIG. 6. Generally, a wild-type ITR comprises a 125 nucleotide region that self-anneals to form a palindromic double-stranded T-shaped, hairpin structure consisting of two cross arms (formed by sequences referred to as B/B′ and C/C′, respectively), a longer stem region (formed by sequences A/A′), and a single-stranded terminal region referred to as the “D” region. (FIG. 6). Generally, the “D” region of an ITR is positioned between the stem region formed by the A/A′ sequences and the insert containing the transgene of the rAAV vector (e.g., positioned on the “inside” of the ITR relative to the terminus of the ITR or proximal to the transgene insert or expression construct of the rAAV vector). In some embodiments, a “D” region comprises the sequence set forth in SEQ ID NO: 23. The “D” region has been observed to play an important role in encapsidation of rAAV vectors by capsid proteins, for example as disclosed by Ling et al. (2015) J Mol Genet Med 9(3).

The disclosure is based, in part, on the surprising discovery that rAAV vectors comprising a “D” region located on the “outside” of the ITR (e.g., proximal to the terminus of the ITR relative to the transgene insert or expression construct) are efficiently encapsidated by AAV capsid proteins than rAAV vectors having ITRs with unmodified (e.g., wild-type) ITRs In some embodiments, rAAV vectors having a modified “D” sequence (e.g., a “D” sequence in the “outside” position) have reduced toxicity relative to rAAV vectors having wild-type ITR sequences.

In some embodiments, a modified “D” sequence comprises at least one nucleotide substitution relative to a wild-type “D” sequence (e.g., SEQ ID NO: 23). A modified “D” sequence may have at least 1, 2, 3, 4, 5, 6, 7, 8, 9, 10, or more than 10 nucleotide substitutions relative to a wild-type “D” sequence (e.g., SEQ ID NO: 23). In some embodiments, a modified “D” sequence comprises at least 10, 11, 12, 13, 14, 15, 16, 17, 18, or 19 nucleic acid substitutions relative to a wild-type “D” sequence (e.g., SEQ ID NO: 23). In some embodiments, a modified “D” sequence is between about 10% and about 99% (e.g., 10%, 15%, 20%, 25%, 30%, 40%, 45%, 50%, 55%, 60%, 65%, 70%, 75%, 80%, 85%, 90%, 95%, or 99%) identical to a wild-type “D” sequence (e.g., SEQ ID NO: 23). In some embodiments, a modified “D” sequence comprises the sequence set forth in SEQ ID NO: 22, also referred to as an “S” sequence as described in Wang et al. (1995) J Mol Biol 250(5):573-80.

An isolated nucleic acid or rAAV vector as described by the disclosure may further comprise a “TRY” sequence, for example as set forth in SEQ ID NO: 24, as described by Francois, et al. 2005. J Virol The Cellular TATA Binding Protein Is Required for Rep-Dependent Replication of a Minimal Adeno-Associated Virus Type 2 p5 Element. In some embodiments, a TRY sequence is positioned between an ITR (e.g., a 5′ ITR) and an expression construct (e.g., a transgene-encoding insert) of an isolated nucleic acid or rAAV vector.

In some aspects, the disclosure relates to Baculovirus vectors comprising an isolated nucleic acid or rAAV vector as described by the disclosure. In some embodiments, the Baculovirus vector is an Autographa californica nuclear polyhedrosis (AcNPV) vector, for example as described by Urabe et al. (2002) Hum Gene Ther 13(16):1935-43 and Smith et al. (2009) Mol Ther 17(11):1888-1896.

In some aspects, the disclosure provides a host cell comprising an isolated nucleic acid or vector as described herein. A host cell can be a prokaryotic cell or a eukaryotic cell. For example, a host cell can be a mammalian cell, bacterial cell, yeast cell, insect cell, etc. In some embodiments, a host cell is a mammalian cell, for example a HEK293T cell. In some embodiments, a host cell is a bacterial cell, for example an E. coli cell.

rAAVs

In some aspects, the disclosure relates to recombinant AAVs (rAAVs) comprising a transgene that encodes a nucleic acid as described herein (e.g., an rAAV vector as described herein). The term “rAAVs” generally refers to viral particles comprising an rAAV vector encapsidated by one or more AAV capsid proteins. An rAAV described by the disclosure may comprise a capsid protein having a serotype selected from AAV1, AAV2, AAV3, AAV4, AAV5, AAV6, AAV7, AAV8, AAV9, and AAV10. In some embodiments, an rAAV comprises a capsid protein from a non-human host, for example a rhesus AAV capsid protein such as AAVrh.10, AAVrh.39, etc. In some embodiments, an rAAV described by the disclosure comprises a capsid protein that is a variant of a wild-type capsid protein, such as a capsid protein variant that includes at least 1, 2, 3, 4, 5, 6, 7, 8, 9, 10, or more than 10 (e.g., 15, 20, 25, 50, 100, etc.) amino acid substitutions (e.g., mutations) relative to the wild-type AAV capsid protein from which it is derived.

In some embodiments, rAAVs described by the disclosure readily spread through the CNS, particularly when introduced into the CSF space or directly into the brain parenchyma. Accordingly, in some embodiments, rAAVs described by the disclosure comprise a capsid protein that is capable of crossing the blood-brain barrier (BBB). For example, in some embodiments, an rAAV comprises a capsid protein having an AAV9 or AAVrh.10 serotype. Production of rAAVs is described, for example, by Samulski et al. (1989) J Virol. 63(9):3822-8 and Wright (2009) Hum Gene Ther. 20(7): 698-706.

In some embodiments, an rAAV as described by the disclosure (e.g., comprising a recombinant rAAV genome encapsidated by AAV capsid proteins to form an rAAV capsid particle) is produced in a Baculovirus vector expression system (BEVS). Production of rAAVs using BEVS are described, for example by Urabe et al. (2002) Hum Gene Ther 13(16):1935-43, Smith et al. (2009) Mol Ther 17(11):1888-1896, U.S. Pat. Nos. 8,945,918, 9,879,282, and International PCT Publication WO 2017/184879. However, an rAAV can be produced using any suitable method (e.g., using recombinant rep and cap genes).

### Pharmaceutical Compositions

In some aspects, the disclosure provides pharmaceutical compositions comprising an isolated nucleic acid or rAAV as described herein and a pharmaceutically acceptable carrier. As used herein, the term “pharmaceutically acceptable” refers to a material, such as a carrier or diluent, which does not abrogate the biological activity or properties of the compound, and is relatively non-toxic, e.g., the material may be administered to an individual without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the patient such that it may perform its intended function. Additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

Compositions (e.g., pharmaceutical compositions) provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

### Methods

The disclosure is based, in part, on compositions for expression of combinations of AD-associated gene products in a subject that act together (e.g., synergistically) to treat Alzheimer's disease. As used herein “treat” or “treating” refers to (a) preventing or delaying onset of Alzheimer's disease; (b) reducing severity of Alzheimer's disease; (c) reducing or preventing development of symptoms characteristic of Alzheimer's disease; (d) and/or preventing worsening of symptoms characteristic of Alzheimer's disease. Symptoms of Alzheimer's disease include, for example, cognitive dysfunction (e.g., dementia, hallucination, memory loss, etc.), motor dysfunction (e.g., difficulty performing daily tasks, etc.), and emotional and behavioral dysfunction.

Accordingly, in some aspects, the disclosure provides a method for treating a subject having or suspected of having Alzheimer's disease, the method comprising administering to the subject a composition (e.g., a composition comprising an isolated nucleic acid or a vector or a rAAV) as described by the disclosure.

A subject is typically a mammal, for example a human, dog, cat, pig, hamster, rat, mouse, etc. In some embodiments, a subject is a human. In some embodiments, a subject is characterized by an APOE4 allele. A subject may be homozygous (e.g., APOE4^{+/+}) or heterozygous for APOE4. In some embodiments, a subject is heterozygous for APOE4 and the second APOE allele of the subject is selected from APOE2 and APOE3.

In some embodiments, a composition is administered directly to the CNS of the subject, for example by direct injection into the brain and/or spinal cord of the subject. Examples of CNS-direct administration modalities include but are not limited to intracerebral injection, intraventricular injection, intracisternal injection, intraparenchymal injection, intrathecal injection, and any combination of the foregoing. In some embodiments, direct injection into the CNS of a subject results in transgene expression (e.g., expression of the first gene product, second gene product, and if applicable, third gene product) in the midbrain, striatum and/or cerebral cortex of the subject. In some embodiments, direct injection into the CNS results in transgene expression (e.g., expression of the first gene product, second gene product, and if applicable, third gene product) in the spinal cord and/or CSF of the subject.

In some embodiments, direct injection to the CNS of a subject comprises convection enhanced delivery (CED). Convection enhanced delivery is a therapeutic strategy that involves surgical exposure of the brain and placement of a small-diameter catheter directly into a target area of the brain, followed by infusion of a therapeutic agent (e.g., a composition or rAAV as described herein) directly to the brain of the subject. CED is described, for example by Debinski et al. (2009) Expert Rev Neurother. 9(10):1519-27.

In some embodiments, a composition is administered peripherally to a subject, for example by peripheral injection. Examples of peripheral injection include subcutaneous injection, intravenous injection, intra-arterial injection, intraperitoneal injection, or any combination of the foregoing. In some embodiments, the peripheral injection is intra-arterial injection, for example injection into the carotid artery of a subject.

In some embodiments, a composition (e.g., a composition comprising an isolated nucleic acid or a vector or a rAAV) as described by the disclosure is administered both peripherally and directly to the CNS of a subject. For example, in some embodiments, a subject is administered a composition by intra-arterial injection (e.g., injection into the carotid artery) and by intraparenchymal injection (e.g., intraparenchymal injection by CED). In some embodiments, the direct injection to the CNS and the peripheral injection are simultaneous (e.g., happen at the same time). In some embodiments, the direct injection occurs prior (e.g., between 1 minute and 1 week, or more before) to the peripheral injection. In some embodiments, the direct injection occurs after (e.g., between 1 minute and 1 week, or more after) the peripheral injection.

The amount of composition (e.g., a composition comprising an isolated nucleic acid or a vector or a rAAV) as described by the disclosure administered to a subject will vary depending on the administration method. For example, in some embodiments, a rAAV as described herein is administered to a subject at a titer between about 10⁹ Genome copies (GC)/kg and about 10¹⁴ GC/kg (e.g., about 10⁹ GC/kg, about 10¹⁰ GC/kg, about 10¹¹ GC/kg, about 10¹² GC/kg, about 10¹² GC/kg, or about 10¹⁴ GC/kg). In some embodiments, a subject is administered a high titer (e.g., >10¹² Genome Copies GC/kg of an rAAV) by injection to the CSF space, or by intraparenchymal injection.

A composition (e.g., a composition comprising an isolated nucleic acid or a vector or a rAAV) as described by the disclosure can be administered to a subject once or multiple times (e.g., 2, 3, 4, 5, 6, 7, 8, 9, 10, 20, or more) times. In some embodiments, a composition is administered to a subject continuously (e.g., chronically), for example via an infusion pump.

### EXAMPLES

### Example 1: Nucleic Acids and rAAV Vectors

This example describes isolated nucleic acids (e.g. vectors, such as rAAV vectors and rAAVs containing isolated nucleic acids) comprising an inhibitory nucleic acid targeting APOE4 and/or a transgene encoding an APOE2 protein or a portion thereof. In some embodiments, constructs described in this example are useful for treating a subject having or suspected of having Alzheimer's disease (AD) who has at least one copy of the APOE4 isoform. In some embodiments, a subject is homozygous for APOE4 isoform (e.g., APO44^{+/+}).

Isolated nucleic acids encoding shRNAs are utilized to knockdown the expression of the APOE4 isoform specifically both in vitro and in vivo. In some embodiments, the shRNAs are non-allele-specific, e.g., they are also capable of knocking down expression of other APOE isoforms (e.g., E2, E3, or E4).

Isolated nucleic acids encoding an APOE2-encoding transgene are utilized overexpress APOE2. The APOE2 transgene is codon-optimized to differ sufficiently from the endogenous APOE2 sequence in cells such that it would not be recognized by shRNAs targeting wild-type APOE, regardless of isoform.

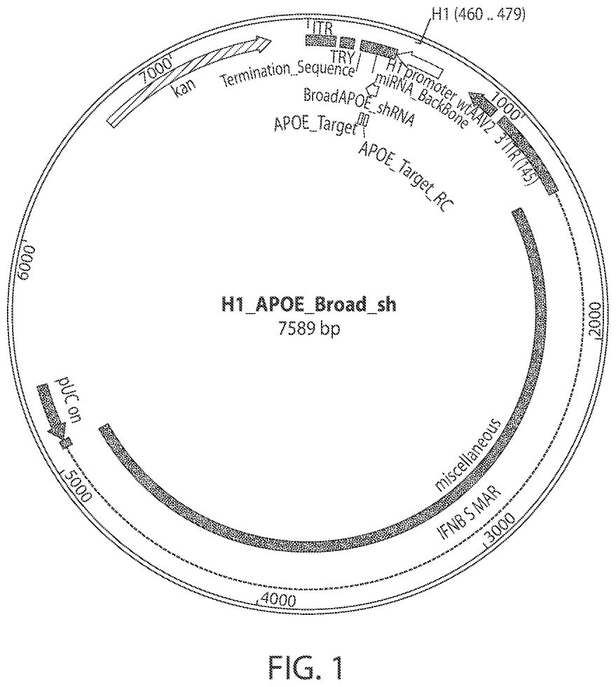

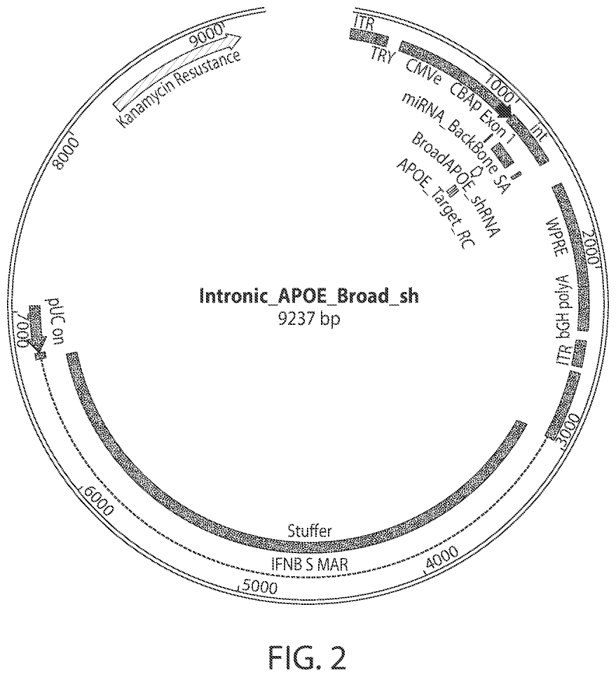

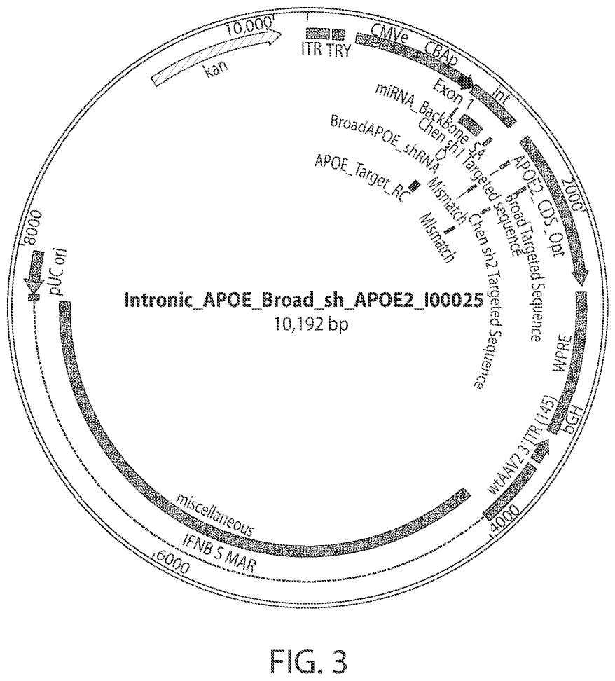

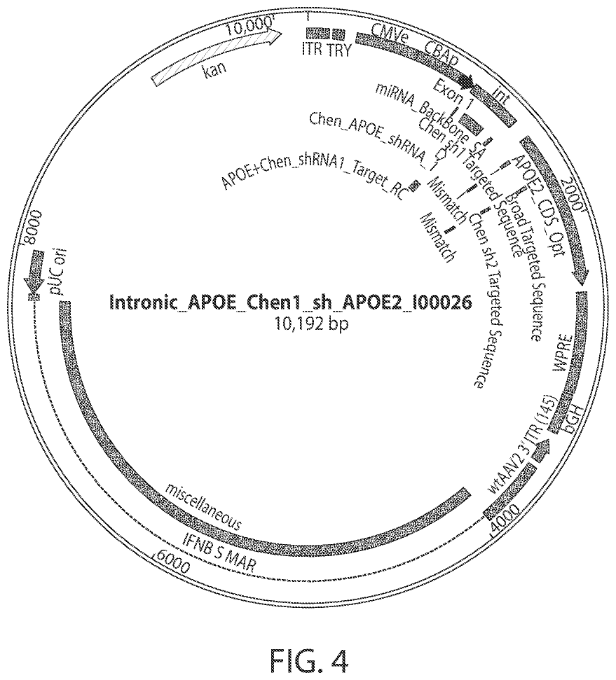

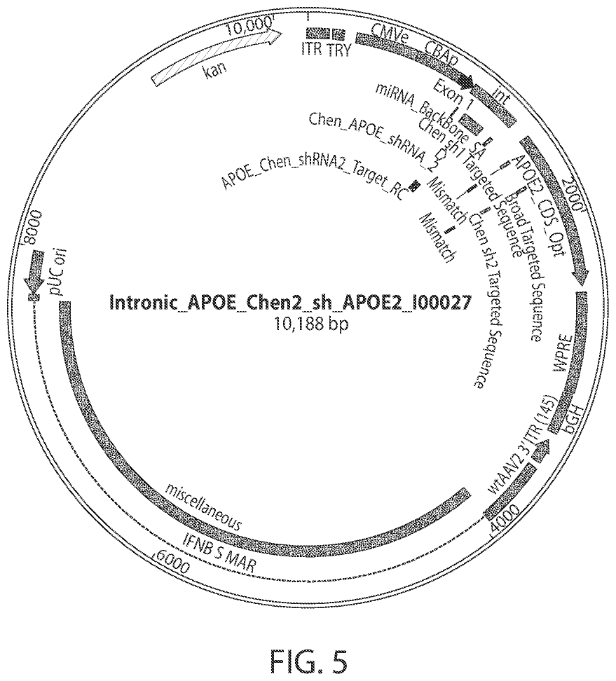

The shRNA and transgene can be operably linked to the same or to separate promoters. shRNAs are expressed under a separate promoter, typically a Pol III promoter (e.g., H1 promoter), or a Pol II promoter (e.g., CBA, T7, etc.). Generally, the shRNA is operably-linked to a Pol II promoter placed in an intronic sequence upstream of an open reading frame comprising the codon-optimized APOE2 transgene. Examples of expression constructs described by the disclosure are shown in FIGS. 1-5 and in Table 1 below.

Recombinant adeno-associated viruses (rAAVs) comprising the isolated nucleic acids are generated using cells, such as HEK293 cells for triple-plasmid transfection. The ITR sequences flank an expression construct, which typically comprises one or more of the following: at least one promoter/enhancer element, a 3′ polyA signal, and posttranslational signals such as the WPRE element. Multiple gene products are expressed simultaneously such as the APOE2 isoform of APOE and one or more inhibitory nucleic acids (e.g., inhibitory nucleic acids targeting the APOE4 isoform of APOE). The presence of a short intronic sequence that is efficiently spliced, upstream of the expressed gene, can improve expression levels. shRNAs and other regulatory RNAs can potentially be included within these sequences.

TABLE 1

| | | | | | Length |
|---|---|---|---|---|---|
| | | | | | between |
| Name | Promoter 1 | shRNA | CDS1 | PolyA 1 | ITRs |
| H1_ApoE_Broad_sh | H1 | ApoE | — | — | 903 |
| Intronic_ApoE_Broad_sh | CMVe_CBAp | ApoE | — | WPRE_bGHpolyA | 2251 |
| Intronic_ApoE_sh_ApoE2_I00025 | CMVe_CBAp | ApoE | APOE2_CDS_opt | WPRE_bGHpolyA | 3507 |
| Intronic_ApoE_Chen1_sh_APOE2_I00026 | CMVe_CBAp | ApoE | ApoE2_CDS_opt | WPRE_bGHpolyA | 3506 |
| Intronic_APOE_Chen2_sh_APOE2_I00027 | CMVe_CBAp | ApoE | ApoE2_CDS_opt | WPRE_bGHpolyA | 3502 |

### Example 2: Cell Based Assays of Viral Transduction into APOE4^{+/+} Cells

Cells are obtained, for example as fibroblasts from AD patients, monocytes, or hES cells, or patient-derived induced pluripotent stem cells (iPSCs). These cells accumulate proteinaceous plaques comprising amyloid-β protein and tangles comprising twisted strands of the protein Tau.

Using such cell models, neurodegenerative characteristics associated with AD are quantified in terms of accumulation of protein aggregates such as plaques and tangles, for example, utilizing an α-amyloid-β antibody or α-phospho-Tau antibody, followed by imaging using fluorescent microscopy. Imaging for neurodegenerative characteristics associated with AD by ICC for protein markers such as amyloid-β, phospho-Tau, or APOE4 is also performed. Western blotting, ELISA, and/or qPCR is used to quantify APOE4 expression levels in these cells.

Therapeutic endpoints (e.g., reduction of AD-associated pathology) are measured in the context of expression of transduction of the rAAVs, to confirm and quantify activity and function. The levels of amyloid-β and phospho-Tau are also quantified using Western blotting, ELISA, and/or qPCR.

### Example 3: In Vivo Assays Using Mice Expressing Human APOE4

This example describes in vivo assays of rAAVs using mutant mice. In vivo studies of rAAVs as above in mutant mice are performed using assays described, for example, by Liao et al. (2018) J. Clin. Invest 128(5): 2144-2155; Rosenberg, et al. (2018) Hum Gene Ther Clin Dev 29(1): 24-47; Zhao et al. (2016) Neurobiol Aging 44: 159-172. These mutant mice harbor the human APOE4 isoform at the murine APOE locus.

The intrathecal or intraventricular delivery of vehicle control and rAAVs (e.g., at a dose of 2×10¹¹ vg/mouse) are performed using concentrated rAAV stocks, for example at an injection volume between 5-10 μL. Intraparenchymal delivery by convection enhanced delivery is performed.

Treatment is initiated either before onset of symptoms, or subsequent to onset. Endpoints measured are the levels of APOE4 and APOE2 expression in the CNS and CSF.

### Example 4: In Vivo Assays of Mouse Models of AD

This example describes in vivo assays of rAAVs using mutant mice. In vivo studies of rAAVs as above in mutant mice are performed using assays described, for example, by Liao et al. (2018) J. Clin. Invest 128(5): 2144-2155; Rosenberg, et al. (2018) Hum Gene Ther Clin Dev 29(1): 24-47; Zhao et al. (2016) Neurobiol Aging 44: 159-172. These mutant mice harbor the human APOE4 isoform at the murine APOE locus. In some instances these mice also express mutant human amyloid precursor protein (APP), mutant human presenilin 1 (PS1) protein, and/or mutant human presenilin 2 (PS2) protein to model the development of amyloid-β plaques in human AD.

Intrathecal or intraventricular delivery of vehicle control and rAAVs (e.g., at a dose of 2×10¹¹ vg/mouse) are performed using concentrated rAAV stocks, for example with injection volume between 5-10 μL. Intraparenchymal delivery by convection enhanced delivery is performed. Peripheral delivery is achieved by tail vein injection.

Treatment is initiated either before onset of symptoms, or subsequent to onset. Endpoints measured are the levels of APOE4 and APOE2 expression in the CNS and CSF, the accumulation longer amyloid-β (Aβ) species, such as Aβ₄₂, an increase in all Aβ species, motor and cognitive endpoints, and accumulation of amyloid-β plaques and Tau tangles.

### Example 5: Clinical Trials in AD Patients

This example describes clinical trials to assess the safety and efficacy of rAAVs as described by the disclosure, in patients having AD.

Clinical trials of rAAVs of the present disclosure for treatment of AD are performed using a study design similar to that described in Grabowski et al. (1995) Ann. Intern. Med. 122(1):33-39. The rAAVs are delivered into the CSF, intraparenchymally to the hippocampus or to another brain region, or peripherally.

Endpoints measured are levels of amyloid-β plaques, Tau tangles, motor and cognitive endpoints, and levels of APOE4 and APOE2 proteins.

### Example 6: Clinical Trials in AD Patients Combined with Amyloid-β Antibodies

This example describes clinical trials to assess the safety and efficacy of rAAVs as described by the disclosure, utilized in combination with amyloid-β antibodies (e.g., bapineuzumab and solanezumab) in patients having AD.

Clinical trials of rAAVs of the present disclosure, combined with anti-amyloid-β antibodies, for treatment of AD are performed using a study design similar to that described in Grabowski et al. (1995) Ann. Intern. Med. 122(1):33-39. The rAAVs are delivered into the CSF, intraparenchymally to the hippocampus or to another brain region, or peripherally.

In some embodiments, rAAVs of the disclosure synergize with anti-amyloid-β antibodies to reduce the likelihood of AD patients developing amyloid-related imaging abnormalities (ARIA), which are highly correlated with APOE genotype. ARIAs are a spectrum of abnormalities observed in AD patients which are associated with amyloid-modifying therapies, particularly with human monoclonal antibodies. There are two types of ARIAs, ARIA-E, which refers to cerebral edema, and ARIA-H, which refers to cerebral microhemmorhages.

Endpoints evaluated are brain imaging before and after treatment to determine if ARIA has occurred and whether rAAVs of the disclosure reduce the likelihood of ARIA, levels of amyloid-β plaques, Tau tangles, motor and cognitive endpoints, and levels of APOE4 and APOE2 proteins.

### Example 7: Clinical Trials in AD Patients which are APOE4^{+/+}, APOE4^{+/-}, and APOE4^{-/-}

This example describes clinical trials to assess the efficacy of rAAVs as described by the disclosure in ameliorating the increased risk of other pathologies including stroke, coronary artery disease, atherosclerosis, poor recovery from head trauma, and cognitive recovery from surgery on a bypass machine, in patients having AD who are APOE4^{+/+} compared to patients who are APOE4^{+/-} or APOE4^{-/-}.

Clinical trials of rAAVs of the disclosure for treatment of AD and ameliorating increased risk of other conditions associated with patients who are APOE4^{+/+} are performed using a study design similar to that described in Grabowski et al. (1995) Ann. Intern. Med. 122(1):33-39. The rAAVs are delivered into the CSF, intraparenchymally to the hippocampus or to another brain region, or peripherally.

Endpoints evaluated before and after treatment with rAAVs of the disclosure are blood pressure, blood cholesterol and blood sugar levels, motor and cognitive endpoints, MRI, PET, and ultrasound imaging of the coronary arteries, recovery from cognitive trauma, and recovery from surgery on a bypass machine.

### Example 8: Prevention of AD or Treatment of AD in Patient Carriers of the APOE4 Isoform

This example describes clinical trials to assess the efficacy of rAAVs as described by the disclosure in reducing the risk of subjects having at least one APOE4 isoform developing AD and in treating AD in patients with at least one APOE4 isoform. Patients with the APOE4 isoform can be either APOE4^{+/+} or APOE4^{+/-}.

Clinical trials of rAAVs of the present disclosure for the prevention or treatment of AD in carriers of the APOE4 allele are performed using a study design similar to that described in Grabowski et al. (1995) Ann. Intern. Med. 122(1):33-39. The rAAVs are delivered into the CSF, intraparenchymally to the hippocampus or to another brain region, or peripherally.

Endpoints evaluated before and after treatment with rAAVs of the present disclosure are the levels of APOE4 and APOE2 in the CSF and the blood and cognitive and motor endpoints.

### Example 9: Testing of ITR “D” Sequence Placement and Cell Transduction

The effect of placement of ITR “D” sequence on cell transduction of rAAVs is investigated. HEK293 cells are transduced with ApoE2-encoding rAAVs having 1) wild-type ITRs (e.g., “D” sequences proximal to the transgene insert and distal to the terminus of the ITR) or 2) ITRs with the “D” sequence located on the “outside” of the vector (e.g., “D” sequence located proximal to the terminus of the ITR and distal to the transgene insert), as shown in FIG. 6.

### Example 9: In Vitro Validation of shRNAs for Endogenous APOE Silencing and APOE2 Over Expression

Multiple plasmids containing unique shRNAs against APOE and codon-optimized coding sequence of APOE2 were evaluated in in vitro transfection screens to assess the extent of APOE (e.g., APOE4) knockdown and heterologous expression of APOE2. Plasmids were specifically designed to selectively knock down the endogenous APOE gene without affecting vector-encoded APOE2 protein expression. Multiple plasmids show reduction of endogenous APOE (FIG. 7A) and expression of codon-optimized APOE2 (FIG. 7B) via qRT-PCR. The shRNA candidates showed significant reduction of endogenous APOE without affecting the codon optimized APOE2.

### Example 10: In Vivo Validation of shRNAs for Endogenous APOE Silencing and APOE2 Over Expression

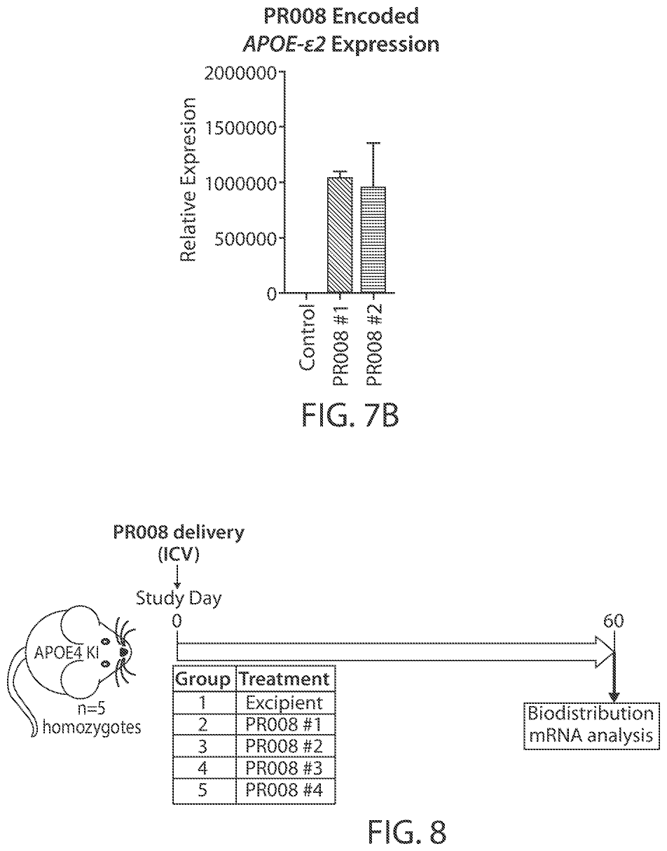

The shRNA candidates demonstrating significant reduction of endogenous APOE without affecting the codon optimized APOE2 are selected for further in vivo study. APOE4 knock-in (KI) mouse model is used to evaluate the in vivo efficacy of the candidate shRNAs against APOE4. In the APOE4 KI mice, both mouse Apoe alleles are replaced by human APOE-ε4. The mice (n=5) receive vectors carrying the candidate shRNAs against APOE4 via intracerebroventricular injection (ICV) and the biodistribution of human APOE4 mRNA is analyzed 60 days post injection (FIG. 8)

### EQUIVALENTS

Having thus described several aspects of at least one embodiment of this invention, it is to be appreciated that various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

It should also be understood that, unless clearly indicated to the contrary, in any methods claimed herein that include more than one step or act, the order of the steps or acts of the method is not necessarily limited to the order in which the steps or acts of the method are recited.

### SEQUENCES

In some embodiments, an expression cassette encoding one or more gene products (e.g., a first, second and/or third gene product) comprises or consists of a sequence set forth in any one of SEQ ID NOs: 1-21. In some embodiments, a gene product is encoded by a portion (e.g., fragment) of a sequence set forth in any one of SEQ ID NOs: 1-21. The skilled artisan recognizes that nucleic acid sequences encoding inhibitory nucleic acids may describe a sequence where all “T” have been replaced with “U”.

>human APOE4 nucleic acid sequence (NM_001302690.1)
(SEQ ID NO: 1)
GGATGGGGAGATAAGAGAAGACCAGGAGGGAGTTAAATAGGGAATGGGTTGGGGG
CGGCTTGGTAAATGTGCTGGGATTAGGCTGTTGCAGATAATGCAACAAGGCTTGGA
AGGCTAACCTGGGACTGGCCAATCACAGGCAGGAAGATGAAGGTTCTGTGGGCTGC
GTTGCTGGTCACATTCCTGGCAGGATGCCAGGCCAAGGTGGAGCAAGCGGTGGAGA
CAGAGCCGGAGCCCGAGCTGCGCCAGCAGACCGAGTGGCAGAGCGGCCAGCGCTG
GGAACTGGCACTGGGTCGCTTTTGGGATTACCTGCGCTGGGTGCAGACACTGTCTGA
GCAGGTGCAGGAGGAGCTGCTCAGCTCCCAGGTCACCCAGGAACTGAGGGCGCTGA
TGGACGAGACCATGAAGGAGTTGAAGGCCTACAAATCGGAACTGGAGGAACAACT
GACCCCGGTGGCGGAGGAGACGCGGGCACGGCTGTCCAAGGAGCTGCAGGCGGCG
CAGGCCCGGCTGGGCGCGGACATGGAGGACGTGTGCGGCCGCCTGGTGCAGTACCG
CGGCGAGGTGCAGGCCATGCTCGGCCAGAGCACCGAGGAGCTGCGGGTGCGCCTCG
CCTCCCACCTGCGCAAGCTGCGTAAGCGGCTCCTCCGCGATGCCGATGACCTGCAG
AAGCGCCTGGCAGTGTACCAGGCCGGGGCCCGCGAGGGCGCCGAGCGCGGCCTCA
GCGCCATCCGCGAGCGCCTGGGGCCCCTGGTGGAACAGGGCCGCGTGCGGGCCGCC
ACTGTGGCTCCCTGGCCGGCCAGCCGCTACAGGAGCGGGCCCAGGCCTGGGGCGAG
CGGCTGCGCGCGCGGATGGAGGAGATGGGCAGCCGGACCCGCGACCGCCTGGACG
AGGTGAAGGAGCAGGTGGCGGAGGTGCGCGCCAAGCTGGAGGAGCAGGCCCAGCA
GATACGCCTGCAGGCCGAGGCCTTCCAGGCCCGCCTCAAGAGCTGGTTCGAGCCCC
TGGTGGAAGACATGCAGCGCCAGTGGGCCGGGCTGGTGGAGAAGGTGCAGGCTGC
CGTGGGCACCAGCGCCGCCCCTGTGCCCAGCGACAATCACTGAACGCCGAAGCCTG
CAGCCATGCGACCCCACGCCACCCCGTGCCTCCTGCCTCCGCGCAGCCTGCAGCGG
GAGACCCTGTCCCCGCCCCAGCCGTCCTCCTGGGGTGGACCCTAGTTTAATAAAGAT
TCACCAAGTTTCACGCATCAAAAAA
>human APOE2 nucleic acid sequence (NM_000041.3)
(SEQ ID NO: 2)
GGGACAGGGGGAGCCCTATAATTGGACAAGTCTGGGATCCTTGAGTCCTACTCAGC
CCCAGCGGAGGTGAAGGACGTCCTTCCCCAGGAGCCGACTGGCCAATCACAGGCAG
GAAGATGAAGGTTCTGTGGGCTGCGTTGCTGGTCACATTCCTGGCAGGATGCCAGG
CCAAGGTGGAGCAAGCGGTGGAGACAGAGCCGGAGCCCGAGCTGCGCCAGCAGAC
CGAGTGGCAGAGCGGCCAGCGCTGGGAACTGGCACTGGGTCGCTTTTGGGATTACC
TGCGCTGGGTGCAGACACTGTCTGAGCAGGTGCAGGAGGAGCTGCTCAGCTCCCAG
GTCACCCAGGAACTGAGGGCGCTGATGGACGAGACCATGAAGGAGTTGAAGGCCT
ACAAATCGGAACTGGAGGAACAACTGACCCCGGTGGCGGAGGAGACGCGGGCACG
GCTGTCCAAGGAGCTGCAGGCGGCGCAGGCCCGGCTGGGCGCGGACATGGAGGAC
GTGTGCGGCCGCCTGGTGCAGTACCGCGGCGAGGTGCAGGCCATGCTCGGCCAGAG
CACCGAGGAGCTGCGGGTGCGCCTCGCCTCCCACCTGCGCAAGCTGCGTAAGCGGC
TCCTCCGCGATGCCGATGACCTGCAGAAGCGCCTGGCAGTGTACCAGGCCGGGGCC
CGCGAGGGCGCCGAGCGCGGCCTCAGCGCCATCCGCGAGCGCCTGGGGCCCCTGGT
GGAACAGGGCCGCGTGCGGGCCGCCACTGTGGGCTCCCTGGCCGGCCAGCCGCTAC
AGGAGCGGGCCCAGGCCTGGGGCGAGCGGCTGCGCGCGCGGATGGAGGAGATGGG
CAGCCGGACCCGCGACCGCCTGGACGAGGTGAAGGAGCAGGTGGCGGAGGTGCGC
GCCAAGCTGGAGGAGCAGGCCCAGCAGATACGCCTGCAGGCCGAGGCCTTCCAGG
CCCGCCTCAAGAGCTGGTTCGAGCCCCTGGTGGAAGACATGCAGCGCCAGTGGGCC
GGGCTGGTGGAGAAGGTGCAGGCTGCCGTGGGCACCAGCGCCGCCCCTGTGCCCAG
CGACAATCACTGAACGCCGAAGCCTGCAGCCATGCGACCCCACGCCACCCCGTGCC
TCCTGCCTCCGCGCAGCCTGCAGCGGGAGACCCTGTCCCCGCCCCAGCCGTCCTCCT
GGGGTGGACCCTAGTTTAATAAAGATTCACCAAGTTTCACGCATCAAAAA
>Human ApoE2 amino acid sequence
(SEQ ID NO: 3)
MKVLWAALLVTFLAGCQAKVEQAVETEPEPELRQQTEWQSGQRWELALGRFWDYLR
WVQTLSEQVQEELLSSQVTQELRALMDETMKELKAYKSELEEQLTPVAEETRARLSKE
LQAAQARLGADMEDVCGRLVQYRGEVQAMLGQSTEELRVRLASHLRKLRKRLLRDA
DDLQKCLAVYQAGAREGAERGLSAIRERLGPLVEQGRVRAATVGSLAGQPLQERAQA
WGERLRARMEEMGSRTRDRLDEVKEQVAEVRAKLEEQAQQIRLQAEAFQARLKSWFE
PLVEDMQRQWAGLVEKVQAAVGTSAAPVPSDNH
>ApoE2 nucleic acid sequence (codon optimized)
(SEQ ID NO: 4)
ATGAAGGTGCTGTGGGCCGCCCTGCTGGTGACCTTCCTGGCCGGCTGCCAGGCCAAa
GTcGAaCAGGCCGTcGAGACCGAGCCCGAGCCCGAGCTGCGCCAGCAGACCGAGTG
GCAGAGCGGCCAGCGCTGGGAGCTGGCCCTGGGCCGCTTCTGGGACTACCTGCGCT
GGGTGCAGACCCTGAGCGAGCAGGTGCAGGAGGAGCTGCTGAGCAGCCAGGTGAC
CCAGGAGCTGCGCGCCCTGATGGACGAGACCATGAAaGAaCTcAAaGCtTAtAAGAGC
GAGCTGGAGGAGCAGCTGACCCCCGTGGCCGAGGAGACCCGCGCCCGCCTGAGCA
AGGAGCTGCAGGCCGCCCAGGCCCGCCTGGGCGCCGACATGGAGGACGTGTGCGG
CCGCCTGGTGCAGTACCGCGGCGAGGTGCAGGCCATGCTGGGCCAGAGCACCGAGG
AGCTGCGCGTGCGCCTGGCCAGCCACCTGCGCAAGCTGCGCAAGCGCCTGCTGCGC
GACGCCGACGACCTGCAGAAGTGCCTGGCCGTGTACCAGGCCGGCGCCCGCGAGGG
CGCCGAGCGCGGCCTGAGCGCCATCCGCGAGCGCCTGGGCCCCCTGGTGGAGCAGG
GCCGCGTGCGCGCCGCCACCGTGGGCAGCCTGGCCGGCCAGCCCCTGCAGGAGCGC
GCCCAGGCCTGGGGCGAGCGCCTGCGCGCCCGCATGGAGGAGATGGGCAGCCGCA
CCCGCGACCGCCTGGACGAGGTGAAGGAGCAGGTGGCCGAGGTGCGCGCCAAGCT
GGAGGAGCAGGCCCAGCAGATCCGCCTGCAGGCCGAGGCCTTCCAGGCCCGCCTGA
AGAGCTGGTTCGAGCCCCTGGTGGAGGACATGCAGCGCCAGTGGGCCGGCCTGGTG
GAGAAGGTGCAGGCCGCCGTGGGCACCAGCGCCGCCCCCGTGCCCAGCGACAACC
ACTAA
>ApoE4 shRNA 1 nucleic acid sequence
(SEQ ID NO: 5)
TTGTAGGCCTTCAACTCCTTC
>ApoE4 shRNA 1 nucleic acid sequence
(SEQ ID NO: 6)
GAAGGAGTTGAAGGCCTACAA
>ApoE4 shRNA 1 with loop
(SEQ ID NO: 7)
TTGTAGGCCTTCAACTCCTTCCATCTGTGGCTTCACTGAAGGAGTTGAAGGCCTACA
A
>ApoE4 amiRNA 1
(SEQ ID NO: 8)
ttgtcatcctcccacggtggccatttgttccatgtgagtgctagtaacaggccttgtgtcctTTGTAGGCCTTCAACTCCTTC
CATCTGTGGCTTCACTGAAGGAGTTGAAGGCCTACAAgacaacagcatacagccttcagcaagcctcc
a
>ApoE4 amiRNA rAAV vector H1
(SEQ ID NO: 9)
ttggccactccctctctgcgcgctcgctcgctcactgaggccgggcgaccaaaggtcgcccgacgcccgggctttgcccgggcggcctc
agtgagcgagcgagcgcgcagagagggagtggccaactccatcactaggggttcctgctagctctgggtatttaagcccgagtgagcac
gcagggtctccattttgaagcgggaggttacgcgttcgtcgactactagtgggtaccagagcaaaaaaattgtcatcctcccacggtggcca
tttgttccatgtgagtgctagtaacaggccttgtgtcctTTGTAGGCCTTCAACTCCTTCCATCTGTGGCTTCA
CTGAAGGAGTTGAAGGCCTACAAgacaaagcatacagccttcagcaagcctccagtggtctcatacagaacttata
agattcccaaatccaaagacatttcacgtttatggtgatttcccagaacacatagcgacatgcaaatattgcagggcgccactcccctgtccct
cacagccatcttcctgccagggcgcacgcgcgctgggtgttcccgcctagtgacactgggcccgcgattccttggagcgggttgatgacg
tcagcgtttcccatggtgaagcttggatctgatccctaggttctagaaccggtgaccaattgttaattaagtttaaaccctcgaggccgcaagc
agatccacgataacaaacagcttttttggggtgaacatattgactgaattccctgcaggttggccactccctctctgcgcgctcgctcgctcac
tgaggccgcccgggcaaagcccgggcgtcgggcgacctttggtcgcccggcctcagtgagcgagcgagcgcgcagagagggagtgg
ccaactccatcactaggggttcct
>ApoE4 amiRNA rAAV vector 1
(SEQ ID NO: 10)
ttggccactccctctctgcgcgctcgctcgctcactgaggccgCCCGGGCAAAGCCCGGGCGTCGGGCGACC
TTTGGTCGCCcggcctcagtgagcgagcgagcgcgcagagagggagtggccaactccatcactaggggttcctgctagctctg
ggtatttaagcccgagtgagcacgcagggtctccattttgaagcgggaggttacgcgttcgtcgactactagtgggtaccagagctccctag
gttctagaaccggtgacgtctcccatggtgaagcttggatctgaattcggtacctagttattaatagtaatcaattacggggtcattagttcatag
cccatatatggagttccgcgttacataacttacggtaaatggcccgcctggctgaccgcccaacgacccccgcccattgacgtcaataatga
cgtatgttcccatagtaacgccaatagggactttccattgacgtcaatgggtggagtatttacggtaaactgcccacttggcagtacatcaagt
gtatcatatgccaagtacgccccctattgacgtcaatgacggtaaatggcccgcctggcattatgcccagtacatgaccttatgggactttcct
acttggcagtacatctacgtattagtcatcgctattaccatggtcgaggtgagccccacgttctgcttcactctccccatctcccccccctcccc
acccccaattttgtatttatttattttttaattattttgtgcagcgatgggggcggggggggggggggggcgcgcgccaggcggggcgggg
cggggcgaggggcggggcggggcgaggcggagaggtgcggcggcagccaatcagagcggcgcgctccgaaagtttccttttatggc
gaggcggcggcggcggcggccctataaaaagcgaagcgcgcggcgggcgggagtcgctgcgacgctgccttcgccccgtgccccg
ctccgccgccgcctcgcgccgcccgccccggctctgactgaccgcgttactcccacaggtgagcgggcgggacggcccttctcctcAg
CgctgtaattagcgcttggtttaatgacggcttgttggaggcttgctgaaggctgtatgctgttgtcTTGTAGGCCTTCAACTC
CTTCAGTGAAGCCACAGATGGAAGGAGTTGAAGGCCTACAAaggacacaaggcctgttactagca
ctcacatggaacaaatggccaccgtgggaggatgacaatttctgtggctgcgtgaaagccttgaggggctccgggagctagagcctctgc
taaccatgttcatgccttcttctttttcctacagctcctgggcaacgtgctggttattgtgctgtctcatcattttggcaaagaattcctcgaagatc
cgaagggaaagtcttccacgactgtgggatccgttcgaagatatcaccggttgagccacccaattgttaattaagtttaaaccctcgaggcc
gcaagcttatcgataatcaacctctggattacaaaatttgtgaaagattgactggtattcttaactatgttgctccttttacgctatgtggatacgct
gctttaatgcctttgtatcatgctattgcttcccgtatggctttcattttctcctccttgtataaatcctggttgctgtctctttatgaggagttgtggcc
cgttgtcaggcaacgtggcgtggtgtgcactgtgtttgctgacgcaacccccactggttggggcattgccaccacctgtcagctcctttccg
ggactttcgctttccccctccctattgccacggcggaactcatcgccgcctgccttgcccgctgctggacaggggctcggctgttgggcact
gacaattccgtggtgttgtcggggaaatcatcgtcctttccttggctgctcgcctgtgttgccacctggattctgcgcgggacgtccttctgcta
cgtcccttcggccctcaatccagcggaccttccttcccgcggcctgctgccggctctgcggcctcttccgcgtcttcgccttcgccctcagac
gagtcggatctccctttgggccgcctccccgcatcgataccgtcgactagagctcgctgatcagcctcgactgtgccttctagttgccagcc
atctgttgtttgcccctcccccgtgccttccttgaccctggaaggtgccactcccactgtcctttcctaataaaatgaggaaattgcatcgcattg
tctgagtaggtgtcattctattctggggggtggggtggggcaggacagcaagggggaggattgggaagacaatagcaggcatgctgggg
agagatccacgataacaaacagcttttttggggtgaacatattgactgaattccctgcaggttggccactccctctctgcgcgctcgctcgctc
actgaggccgcccgggcaaagcccgggcgtcgggcgacctttggtcgcccggcctcagtgagcgagcgagcgcgcagagagggagt
ggccaactccatcactaggggttcct
>ApoE4 amiRNA-ApoE2opt rAAV vector 1
(SEQ ID NO: 11)
ttggccactccctctctgcgcgctcgctcgctcactgaggccgggcgaccaaaggtcgcccgacgcccgggctttgcccgggcggcctc
agtgagcgagcgagcgcgcagagagggagtggccaactccatcactaggggttcctgctagctctgggtatttaagcccgagtgagcac
gcagggtctccattttgaagcgggaggttacgcgttcgtcgactactagtgggtaccagagctccctaggttctagaaccggtgacgtctcc
catggtgaagcttggatctgaattcggtaccctagttattaatagtaatcaattacggggtcattagttcatagcccatatatggagttccgcgtt
acataacttacggtaaatggcccgcctggctgaccgcccaacgacccccgcccattgacgtcaataatgacgtatgttcccatagtaacgc
caatagggactttccattgacgtcaatgggtggactatttacggtaaactgcccacttggcagtacatcaagtgtatcatatgccaagtacgcc
ccctattgacgtcaatgacggtaaatggcccgcctggcattatgcccagtacatgaccttatgggactttcctacttggcagtacatctacgtat
tagtcatcgctattaccatggtcgaggtgagccccacgttctgcttcactctccccatctcccccccctccccacccccaattttgtatttatttat
tttttaattattttgtgcagcgatgggggcggggggggggggggggcgcgcgccaggcggggcggggcggggcgaggggcggggc
ggggcgaggcggagaggtgcggcggcagccaatcagagcggcgcgctccgaaagtttccttttatggcgaggcggcggcggcggcg
gccctataaaaagcgaagcgcgcggcgggcgggagtcgctgcgacgctgccttcgccccgtgccccgctccgccgccgcctcgcgcc
gcccgccccggctctgactgaccgcgttactcccacaggtgagcgggcgggacggcccttctcctcAgCgctgtaattagcgcttggttt
aatgacggcttgttggaggcttgctgaaggctgtatgctgttgtcTTGTAGGCCTTCAACTCCTTCAGTGAAGCC
ACAGATGGAAGGAGTTGAAGGCCTACAAaggacacaaggcctgttactagcactcacatggaacaaatggcc
accgtgggaggatgacaatttctgtggctgcgtgaaagccttgaggggctccgggagctagagcctctgctaaccatgttcatgccttcttct
ttttcctacagctcctgggcaacgtgctggttattgtgctgtctcatcattttggcaaagaattcctcgaagatccgaagggaaagtcttccacg
actgtgggatccgttcgaagatatcaccggttgagccaccATGAAGGTGCTGTGGGCCGCCCTGCTGGTGAC
CTTCCTGGCCGGCTGCCAGGCCAAaGTcGAaCAGGCCGTcGAGACCGAGCCCGAGCC
CGAGCTGCGCCAGCAGACCGAGTGGCAGAGCGGCCAGCGCTGGGAGCTGGCCCTG
GGCCGCTTCTGGGACTACCTGCGCTGGGTGCAGACCCTGAGCGAGCAGGTGCAGGA
GGAGCTGCTGAGCAGCCAGGTGACCCAGGAGCTGCGCGCCCTGATGGACGAGACC
ATGAAaGAaCTcAAaGCtTAtAAGAGCGAGCTGGAGGAGCAGCTGACCCCCGTGGCCG
AGGAGACCCGCGCCCGCCTGAGCAAGGAGCTGCAGGCCGCCCAGGCCCGCCTGGG
CGCCGACATGGAGGACGTGTGCGGCCGCCTGGTGCAGTACCGCGGCGAGGTGCAGG
CCATGCTGGGCCAGAGCACCGAGGAGCTGCGCGTGCGCCTGGCCAGCCACCTGCGC
AAGCTGCGCAAGCGCCTGCTGCGCGACGCCGACGACCTGCAGAAGTGCCTGGCCGT
GTACCAGGCCGGCGCCCGCGAGGGCGCCGAGCGCGGCCTGAGCGCCATCCGCGAG
CGCCTGGGCCCCCTGGTGGAGCAGGGCCGCGTGCGCGCCGCCACCGTGGGCAGCCT
GGCCGGCCAGCCCCTGCAGGAGCGCGCCCAGGCCTGGGGCGAGCGCCTGCGCGCCC
GCATGGAGGAGATGGGCAGCCGCACCCGCGACCGCCTGGACGAGGTGAAGGAGCA
GGTGGCCGAGGTGCGCGCCAAGCTGGAGGAGCAGGCCCAGCAGATCCGCCTGCAG
GCCGAGGCCTTCCAGGCCCGCCTGAAGAGCTGGTTCGAGCCCCTGGTGGAGGACAT
GCAGCGCCAGTGGGCCGGCCTGGTGGAGAAGGTGCAGGCCGCCGTGGGCACCAGC
GCCGCCCCCGTGCCCAGCGACAACCACTAAcaattgttaattaagtttaaaccctcgaggccgcaagcttatcg
ataatcaacctctggattacaaaatttgtgaaagattgactggtattcttaactatgttgctccttttacgctatgtggatacgctgctttaatgcctt
tgtatcatgctattgcttcccgtatggctttcattttctcctccttgtataaatcctggttgctgtctctttatgaggagttgtggcccgttgtc aggc a
acgtggcgtggtgtgcactgtgtttgctgacgcaacccccactggttggggcattgccaccacctgtcagctcctttccgggactttcgctttc
cccctccctattgccacggcggaactcatcgccgcctgccttgcccgctgctggacaggggctcggctgttgggcactgacaattccgtgg
tgttgtcggggaaatcatcgtcctttccttggctgctcgcctgtgttgccacctggattctgcgcgggacgtccttctgctacgtcccttcggcc
ctcaatccagcggaccttccttcccgcggcctgctgccggctctgcggcctcttccgcgtcttcgccttcgccctcagacgagtcggatctc
cctttgggccgcctccccgcatcgataccgtcgactagagctcgctgatcagcctcgactgtgccttctagttgccagccatctgttgtttgcc
cctcccccgtgccttccttgaccctggaaggtgccactcccactgtcctttcctaataaaatgaggaaattgcatcgcattgtctgagtaggtgt
cattctattctggggggtggggtggggcaggacagcaagggggaggattgggaagacaatagcaggcatgctggggagagatccacg
ataacaaacagcttttttggggtgaacatattgactgaattccctgcaggttggccactccctctctgcgcgctcgctcgctcactgaggccgc
ccgggcaaagcccgggcgtcgggcgacctttggtcgcccggcctcagtgagcgagcgagcgcgcagagagggagtggccaactcca
tcactaggggttcct
>ApoE4 shRNA 2 nucleic acid sequence
(SEQ ID NO: 12)
ctccaccgcttgctccacctt
>ApoE4 shRNA 2 nucleic acid sequence
(SEQ ID NO: 13)
aaggtggagcaagcggtggag
>ApoE4 shRNA 2 with loop
(SEQ ID NO: 14)
ctccaccgcttgctccaccttAGTGAAGCCACAGATGaaggtggagcaagcggtggag
>ApoE4 amiRNA 2
(SEQ ID NO: 15)
tggaggcttgctgaaggctgtatgctgttgtcctccaccgcttgctccaccttAGTGAAGCCACAGATGaaggtggagcaag
cggtggagaggacacaaggcctgttactagcactcacatggaacaaatggccaccgtgggaggatgacaa
>ApoE4 amiRNA-ApoE2opt rAAV vector 2
(SEQ ID NO: 16)
ttggccactccctctctgcgcgctcgctcgctcactgaggccgggcgaccaaaggtcgcccgacgcccgggctttgcccgggcggcctc
agtgagcgagcgagcgcgcagagagggagtggccaactccatcactaggggttcctgctagctctgggtatttaagcccgagtgagcac
gcagggtctccattttgaagcgggaggttacgcgttcgtcgactactagtgggtaccagagctccctaggttctagaaccggtgacgtctcc
catggtgaagcttggatctgaattcggtaccctagttattaatagtaatcaattacggggtcattagttcatagcccatatatggagttccgcgtt
acataacttacggtaaatggcccgcctggctgaccgcccaacgacccccgcccattgacgtcaataatgacgtatgttcccatagtaacgc
caatagggactttccattgacgtcaatgggtggactatttacggtaaactgcccacttggcagtacatcaagtgtatcatatgccaagtacgcc
ccctattgacgtcaatgacggtaaatggcccgcctggcattatgcccagtacatgaccttatgggactttcctacttggcagtacatctacgtat
tagtcatcgctattaccatggtcgaggtgagccccacgttctgcttcactctccccatctcccccccctccccacccccaattttgtatttatttat
tttttaattattttgtgcagcgatgggggcggggggggggggggggcgcgcgccaggcggggcggggcggggcgaggggcggggc
ggggcgaggcggagaggtgcggcggcagccaatcagagcggcgcgctccgaaagtttccttttatggcgaggcggcggcggcggcg
gccctataaaaagcgaagcgcgcggcgggcgggagtcgctgcgacgctgccttcgccccgtgccccgctccgccgccgcctcgcgcc
gcccgccccggctctgactgaccgcgttactcccacaggtgagcgggcgggacggcccttctcctcAgCgctgtaattagcgcttggttt
aatgacggcttgttggaggcttgctgaaggctgtatgctgttgtcctccaccgcttgctccaccttAGTGAAGCCACAGATGa
aggtggagcaagcggtggagaggacacaaggcctgttactagcactcacatggaacaaatggccaccgtgggaggatgacaatttctgt
ggctgcgtgaaagccttgaggggctccgggagctagagcctctgctaaccatgttcatgccttcttctttttcctacagctcctgggcaacgt
gctggttattgtgctgtctcatcattttggcaaagaattcctcgaagatccgaagggaaagtcttccacgactgtgggatccgttcgaagatat
caccggttgagccaccATGAAGGTGCTGTGGGCCGCCCTGCTGGTGACCTTCCTGGCCGGCT
GCCAGGCCAAaGTcGAaCAGGCCGTcGAGACCGAGCCCGAGCCCGAGCTGCGCCAGC
AGACCGAGTGGCAGAGCGGCCAGCGCTGGGAGCTGGCCCTGGGCCGCTTCTGGGAC
TACCTGCGCTGGGTGCAGACCCTGAGCGAGCAGGTGCAGGAGGAGCTGCTGAGCAG
CCAGGTGACCCAGGAGCTGCGCGCCCTGATGGACGAGACCATGAAaGAaCTcAAaGCt
TAtAAGAGCGAGCTGGAGGAGCAGCTGACCCCCGTGGCCGAGGAGACCCGCGCCCG
CCTGAGCAAGGAGCTGCAGGCCGCCCAGGCCCGCCTGGGCGCCGACATGGAGGAC
GTGTGCGGCCGCCTGGTGCAGTACCGCGGCGAGGTGCAGGCCATGCTGGGCCAGAG
CACCGAGGAGCTGCGCGTGCGCCTGGCCAGCCACCTGCGCAAGCTGCGCAAGCGCC
TGCTGCGCGACGCCGACGACCTGCAGAAGTGCCTGGCCGTGTACCAGGCCGGCGCC
CGCGAGGGCGCCGAGCGCGGCCTGAGCGCCATCCGCGAGCGCCTGGGCCCCCTGGT
GGAGCAGGGCCGCGTGCGCGCCGCCACCGTGGGCAGCCTGGCCGGCCAGCCCCTGC
AGGAGCGCGCCCAGGCCTGGGGCGAGCGCCTGCGCGCCCGCATGGAGGAGATGGG
CAGCCGCACCCGCGACCGCCTGGACGAGGTGAAGGAGCAGGTGGCCGAGGTGCGC
GCCAAGCTGGAGGAGCAGGCCCAGCAGATCCGCCTGCAGGCCGAGGCCTTCCAGGC
CCGCCTGAAGAGCTGGTTCGAGCCCCTGGTGGAGGACATGCAGCGCCAGTGGGCCG
GCCTGGTGGAGAAGGTGCAGGCCGCCGTGGGCACCAGCGCCGCCCCCGTGCCCAGC
GACAACCACTAAcaattgttaattaagtttaaaccctcgaggccgcaagcttatcgataatcaacctctggattacaaaatttgtga
aagattgactggtattcttaactatgttgctccttttacgctatgtggatacgctgctttaatgcctttgtatcatgctattgcttcccgtatggctttc
attttctcctccttgtataaatcctggttgctgtctctttatgaggagttgtggcccgttgtcaggcaacgtggcgtggtgtgcactgtgtttgctg
acgcaacccccactggttggggcattgccaccacctgtcagctcctttccgggactttcgctttccccctccctattgccacggcggaactca
tcgccgcctgccttgcccgctgctggacaggggctcggctgttgggcactgacaattccgtggtgttgtcggggaaatcatcgtcctttcctt
ggctgctcgcctgtgttgccacctggattctgcgcgggacgtccttctgctacgtcccttcggccctcaatccagcggaccttccttcccgcg
gcctgctgccggctctgcggcctcttccgcgtcttcgccttcgccctcagacgagtcggatctccctttgggccgcctccccgcatcgatac
cgtcgactagagctcgctgatcagcctcgactgtgccttctagttgccagccatctgttgtttgcccctcccccgtgccttccttgaccctgga
aggtgccactcccactgtcctttcctaataaaatgaggaaattgcatcgcattgtctgagtaggtgtcattctattctggggggtggggtgggg
caggacagcaagggggaggattgggaagacaatagcaggcatgctggggagagatccacgataacaaacagcttttttggggtgaacat
attgactgaattccctgcaggttggccactccctctctgcgcgctcgctcgctcactgaggccgcccgggcaaagcccgggcgtcgggcg
acctttggtcgcccggcctcagtgagcgagcgagcgcgcagagagggagtggccaactccatcactaggggttcct
>ApoE4 shRNA 3 nucleic acid sequence
(SEQ ID NO: 17)
tttgtaggccttcaactcc
>ApoE4 shRNA 3 nucleic acid sequence
(SEQ ID NO: 18)
ggagttgaaggcctacaaa
>ApoE4 shRNA 3 with loop
(SEQ ID NO: 19)
tttgtaggccttcaactccAGTGAAGCCACAGATGggagttgaaggcctacaaa
>ApoE4 amiRNA 3
(SEQ ID NO: 20)
tggaggcttgctgaaggctgtatgctgttgtctttgtaggccttcaactccAGTGAAGCCACAGATGggagttgaaggcctac
aaaaggacacaaggcctgttactagcactcacatggaacaaatggccaccgtgggaggatgacaa
>ApoE4 amiRNA-ApoE2opt rAAV vector 3
(SEQ ID NO: 21)
ttggccactccctctctgcgcgctcgctcgctcactgaggccgggcgaccaaaggtcgcccgacgcccgggctttgcccgggcggcctc
agtgagcgagcgagcgcgcagagagggagtggccaactccatcactaggggttcctgctagctctgggtatttaagcccgagtgagcac
gcagggtctccattttgaagcgggaggttacgcgttcgtcgactactagtgggtaccagagctccctaggttctagaaccggtgacgtctcc
catggtgaagcttggatctgaattcggtaccctagttattaatagtaatcaattacggggtcattagttcatagcccatatatggagttccgcgtt
acataacttacggtaaatggcccgcctggctgaccgcccaacgacccccgcccattgacgtcaataatgacgtatgttcccatagtaacgc
caatagggactttccattgacgtcaatgggtggactatttacggtaaactgcccacttggcagtacatcaagtgtatcatatgccaagtacgcc
ccctattgacgtcaatgacggtaaatggcccgcctggcattatgcccagtacatgaccttatgggactttcctacttggcagtacatctacgtat
tagtcatcgctattaccatggtcgaggtgagccccacgttctgcttcactctccccatctcccccccctccccacccccaattttgtatttatttat
tttttaattattttgtgcagcgatgggggcggggggggggggggggcgcgcgccaggcggggcggggcggggcgaggggcggggc
ggggcgaggcggagaggtgcggcggcagccaatcagagcggcgcgctccgaaagtttccttttatggcgaggcggcggcggcggcg
gccctataaaaagcgaagcgcgcggcgggcgggagtcgctgcgacgctgccttcgccccgtgccccgctccgccgccgcctcgcgcc
gcccgccccggctctgactgaccgcgttactcccacaggtgagcgggcgggacggcccttctcctcAgCgctgtaattagcgcttggttt
aatgacggcttgttggaggcttgctgaaggctgtatgctgttgtctttgtaggccttcaactccAGTGAAGCCACAGATGgga
gttgaaggcctacaaaaggacacaaggcctgttactagcactcacatggaacaaatggccaccgtgggaggatgacaatttctgtggctgc
gtgaaagccttgaggggctccgggagctagagcctctgctaaccatgttcatgccttcttctttttcctacagctcctgggcaacgtgctggtt
attgtgctgtctcatcattttggcaaagaattcctcgaagatccgaagggaaagtcttccacgactgtgggatccgttcgaagatatcaccggt
tgagccaccATGAAGGTGCTGTGGGCCGCCCTGCTGGTGACCTTCCTGGCCGGCTGCCAG
GCCAAaGTcGAaCAGGCCGTcGAGACCGAGCCCGAGCCCGAGCTGCGCCAGCAGACC
GAGTGGCAGAGCGGCCAGCGCTGGGAGCTGGCCCTGGGCCGCTTCTGGGACTACCT
GCGCTGGGTGCAGACCCTGAGCGAGCAGGTGCAGGAGGAGCTGCTGAGCAGCCAG
GTGACCCAGGAGCTGCGCGCCCTGATGGACGAGACCATGAAaGAaCTcAAaGCtTAtA
AGAGCGAGCTGGAGGAGCAGCTGACCCCCGTGGCCGAGGAGACCCGCGCCCGCCT
GAGCAAGGAGCTGCAGGCCGCCCAGGCCCGCCTGGGCGCCGACATGGAGGACGTG
TGCGGCCGCCTGGTGCAGTACCGCGGCGAGGTGCAGGCCATGCTGGGCCAGAGCAC
CGAGGAGCTGCGCGTGCGCCTGGCCAGCCACCTGCGCAAGCTGCGCAAGCGCCTGC
TGCGCGACGCCGACGACCTGCAGAAGTGCCTGGCCGTGTACCAGGCCGGCGCCCGC
GAGGGCGCCGAGCGCGGCCTGAGCGCCATCCGCGAGCGCCTGGGCCCCCTGGTGGA
GCAGGGCCGCGTGCGCGCCGCCACCGTGGGCAGCCTGGCCGGCCAGCCCCTGCAGG
AGCGCGCCCAGGCCTGGGGCGAGCGCCTGCGCGCCCGCATGGAGGAGATGGGCAG
CCGCACCCGCGACCGCCTGGACGAGGTGAAGGAGCAGGTGGCCGAGGTGCGCGCC
AAGCTGGAGGAGCAGGCCCAGCAGATCCGCCTGCAGGCCGAGGCCTTCCAGGCCCG
CCTGAAGAGCTGGTTCGAGCCCCTGGTGGAGGACATGCAGCGCCAGTGGGCCGGCC
TGGTGGAGAAGGTGCAGGCCGCCGTGGGCACCAGCGCCGCCCCCGTGCCCAGCGAC
AACCACTAAcaattgttaattaagtttaaaccctcgaggccgcaagcttatcgataatcaacctctggattacaaaatttgtgaaagatt
gactggtattcttaactatgttgctccttttacgctatgtggatacgctgctttaatgcctttgtatcatgctattgcttcccgtatggctttcattttctc
ctccttgtataaatcctggttgctgtctctttatgaggagttgtggcccgttgtcaggcaacgtggcgtggtgtgcactgtgtttgctgacgcaa
cccccactggttggggcattgccaccacctgtcagctcctttccgggactttcgctttccccctccctattgccacggcggaactcatcgccg
cctgccttgcccgctgctggacaggggctcggctgttgggcactgacaattccgtggtgttgtcggggaaatcatcgtcctttccttggctgc
tcgcctgtgttgccacctggattctgcgcgggacgtccttctgctacgtcccttcggccctcaatccagcggaccttccttcccgcggcctgc
tgccggctctgcggcctcttccgcgtcttcgccttcgccctcagacgagtcggatctccctttgggccgcctccccgcatcgataccgtcga
ctagagctcgctgatcagcctcgactgtgccttctagttgccagccatctgttgtttgcccctcccccgtgccttccttgaccctggaaggtgc
cactcccactgtcctttcctaataaaatgaggaaattgcatcgcattgtctgagtaggtgtcattctattctggggggtggggtggggcaggac
agcaagggggaggattgggaagacaatagcaggcatgctggggagagatccacgataacaaacagcttttttggggtgaacatattgact
gaattccctgcaggttggccactccctctctgcgcgctcgctcgctcactgaggccgcccgggcaaagcccgggcgtcgggcgacctttg
gtcgcccggcctcagtgagcgagcgagcgcgcagagagggagtggccaactccatcactaggggttcct
>AAV2 ITR D region “S” sequence
(SEQ ID NO: 22)
TATTAGATCTGATGGCCGC
>AAV2 ITR D region “D” sequence
(SEQ ID NO: 23)
CTCCATCACTAGGGGTTCCT
>TRY motif sequence
(SEQ ID NO: 24)
AGCTCTGGGTATTTAAGCCCGAGTGAGCACGCAGGGTCTCCATTTTGAAGCGGGAG
GTTA
>Wild-type AAV2 ITR nucleic acid sequence
(SEQ ID NO: 25)
AGGAACCCCTAGTGATGGAGTTGGCCACTCCCTCTCTGCGCGCTCGCTCGCTCACTG
AGGCCGGGCGACCAAAGGTCGCCCGACGCCCGGGCTTTGCCCGGGCGGCCTCAGTG
AGCGAGCGAGCGCGCAGAGAGGGAGTGGCCAA

What is claimed is:

1. An isolated nucleic acid comprising an expression construct encoding an inhibitory nucleic acid that inhibits expression or activity of APOE4 and a transgene that expresses APOE2.

2. The isolated nucleic acid of claim 1, wherein the inhibitory nucleic acid is encoded by a sequence set forth in any one of SEQ ID NOs: 5-8, 12-15, and 17-20.

3. The isolated nucleic acid of claim 1 or 2, wherein the inhibitory nucleic acid is encoded by the sequence set forth in any one of SEQ ID NOs: 7, 8, 14, 15, 19, and 20.

4. The isolated nucleic acid of any one of claims 1 to 3, wherein the transgene that expresses APOE2 encodes a protein having an amino acid sequence set forth in SEQ ID NO: 3.

5. The isolated nucleic acid of any one of claims 1 to 4, wherein the transgene that expresses APOE2 comprises a codon optimized nucleic acid sequence, optionally wherein the nucleic acid sequence is set forth in SEQ ID NO: 4.

6. The isolated nucleic acid of any one of claims 1 to 5, wherein the expression construct is flanked by adeno-associated virus (AAV) inverted terminal repeats (ITRs).

7. The isolated nucleic acid of claim 6, wherein the ITRs are AAV2 ITRs.

8. The isolated nucleic acid of any one of claims 1 to 7, wherein the isolated nucleic acid comprises the sequence set forth in any one of SEQ ID NOs: 11, 16, and 21.

9. An isolated nucleic acid comprising an expression construct encoding an APOE2 protein, wherein the isolated nucleic acid comprises the sequence set forth in SEQ ID NO: 4.

10. An isolated nucleic acid comprising an expression construct encoding an inhibitory nucleic acid that inhibits expression or activity of APOE4.

11. The isolated nucleic acid of claim 9 or 10, wherein the expression construct is flanked by adeno-associated virus (AAV) inverted terminal repeats (ITRs), optionally wherein the ITRs are AAV2 ITRs.

12. The isolated nucleic acid of any one of claims 1 to 11, further comprising one or more promoters, optionally wherein each of the one or more promoters is independently a chicken-beta actin (CBA) promoter, a CAG promoter, a CD68 promoter, or a JeT promoter.

13. A vector comprising the isolated nucleic acid of any one of claims 1 to 12.

14. The vector of claim 13, wherein the vector is a plasmid.

15. The vector of claim 13, wherein the vector is a viral vector, optionally wherein the viral vector is a recombinant AAV (rAAV) vector or a Baculovirus vector.

16. A composition comprising the isolated nucleic acid of any one of claims 1 to 12 or the vector of any one of claims 13 to 15.

17. A host cell comprising the isolated nucleic acid of any one of claims 1 to 12 or the vector of any one of claims 13 to 15.

18. A recombinant adeno-associated virus (rAAV) comprising:
(i) a capsid protein; and
(ii) the isolated nucleic acid of any one of claims 1 to 12, or the vector of claim 15.

19. The rAAV of claim 18, wherein the capsid protein is capable of crossing the blood-brain barrier, optionally wherein the capsid protein is an AAV9 capsid protein or an AAVrh.10 capsid protein.

20. The rAAV of claim 18 or claim 19, wherein the rAAV transduces neuronal cells and non-neuronal cells of the central nervous system (CNS).

21. A method for treating a subject having or suspected of having Alzheimer's disease, the method comprising administering to the subject an isolated nucleic acid of any one of claims 1 to 12, the vector of any one of claims 13 to 15, the composition of claim 16, or the rAAV of any one of claims 18-20.

22. The method of claim 21, wherein the administration comprises direct injection to the CNS of the subject, optionally wherein the direct injection is intracerebral injection, intraparenchymal injection, intrathecal injection, or any combination thereof.

23. The method of claim 22, wherein the direct injection to the CNS of the subject comprises convection enhanced delivery (CED).

24. The method of any one of claims 21-23, wherein the administration comprises peripheral injection, optionally wherein the peripheral injection is intravenous injection.

25. The method of any one of claims 21-24, wherein the subject is homozygous for APOE4 alleles.
